SEQUENCE LISTING (24 sequences)

SEQ ID NO: 1 - protein, 18 aa, Artificial Sequence
  antimicrobial peptide
GNNRPVYIPQPRPPHPRL

SEQ ID NO: 2 - protein, 8 aa, Artificial Sequence
  antimicrobial peptide
  MOD_RES (8)..(8): AMIDATION
RIWVIWRR

SEQ ID NO: 3 - protein, 11 aa, Artificial Sequence
  antimicrobial peptide
  MOD_RES (11)..(11): AMIDATION
RRLFRRILRWL

SEQ ID NO: 4 - protein, 23 aa, Artificial Sequence
  antimicrobial peptide
GIGKHVGKALKGLKGLLKGLGEC

SEQ ID NO: 5 - protein, 11 aa, Artificial Sequence
  antimicrobial peptide
  MOD_RES (11)..(11): AMIDATION
RIVQRIKKWLR

SEQ ID NO: 6 - protein, 12 aa, Artificial Sequence
  antimicrobial peptide
  MOD_RES (12)..(12): AMIDATION
KRIVQRIKKWLR

SEQ ID NO: 7 - protein, 11 aa, Artificial Sequence
  antimicrobial peptide
  VARIANT (1)..(1): may also be K or A
  MOD_RES (11)..(11): AMIDATION
  VARIANT (11)..(11): may also be absent
RIVQRIKKWLR

SEQ ID NO: 8 - protein, 12 aa, Artificial Sequence
  antimicrobial peptide
  VARIANT (2)..(2): may also be K or A
  VARIANT (12)..(12): may also be absent
  MOD_RES (12)..(12): AMIDATION
KRIVQRIKKWLR

SEQ ID NO: 9 - protein, 18 aa, Artificial Sequence
  AMP from US2015/0344527
RIVQRIKKWLLKWKKLGY

SEQ ID NO: 10 - protein, 8 aa, Artificial Sequence
  sequence was added after Nisin A
PRPPHPRL

SEQ ID NO: 11 - protein, 7 aa, Artificial Sequence
  sequence was added after Nisin A
NGVQPKY

SEQ ID NO: 12 - protein, 10 aa, Artificial Sequence
  sequence was added after ring ABCDE of nisin A
SVNGVQPKYK

SEQ ID NO: 13 - protein, 13 aa, Artificial Sequence
  sequence was added after ing ABCDE of nisin A
SVKIAKVALKALK

SEQ ID NO: 14 - protein, 11 aa, Artificial Sequence
  sequence was added after rind ABCDE of nisin A
SVPRPPHPRLK

SEQ ID NO: 15 - protein, 11 aa, Artificial Sequence
  antimicrobial peptide GNP-8
  MOD_RES (11)..(11): AMIDATION
RIVQRIKKWLR

SEQ ID NO: 16 - protein, 12 aa, Artificial Sequence
  antimicrobial peptide GNP9
  MOD_RES (12)..(12): AMIDATION
KRIVQRIKKWLR

SEQ ID NO: 17 - protein, 10 aa, Artificial Sequence
  antimicrobial peptide GNP8-1
  MOD_RES (10)..(10): AMIDATION
RIVQRIKKWL

SEQ ID NO: 18 - protein, 11 aa, Artificial Sequence
  antimicrobial peptide GNP8-2
  MOD_RES (11)..(11): AMIDATION
KIVQRIKKWLR

SEQ ID NO: 19 - protein, 11 aa, Artificial Sequence
  antimicrobial peptide GNP8-3
  MOD_RES (11)..(11): AMIDATION
AIVQRIKKWLR

SEQ ID NO: 20 - protein, 11 aa, Artificial Sequence
  antimicrobial peptide GNP8-4
  MOD_RES (11)..(11): AMIDATION
RIRKRIKKWLR

SEQ ID NO: 21 - protein, 11 aa, Artificial Sequence
  antimicrobial peptide GNP8-5
  MOD_RES (11)..(11): AMIDATION
RIKRRIKKWLR

SEQ ID NO: 22 - protein, 10 aa, Artificial Sequence
  antimicrobial peptide GNP8-6
  MOD_RES (10)..(10): AMIDATION
RIVQRIKKWR

SEQ ID NO: 23 - protein, 12 aa, Artificial Sequence
  consensus sequence GNP-8/9
  VARIANT (2)..(2): May also be K or A
  MOD_RES (12)..(12): AMIDATION
KRIVQRIKKWLR

SEQ ID NO: 24 - protein, 13 aa, Artificial Sequence
  consensus sequence GNP-8/9
  VARIANT (2)..(2): May also be K or A
  MOD_RES (13)..(13): AMIDATION
KRIVQRIKKWLRR

(19) United States
(12) Patent Application Publication
(10) Pub. No.: US 2020/0368315 A1
(43) Pub. Date: Nov. 26, 2020

(54) ANTIMICROBIAL PEPTIDES AND ADMIXTURES THEREOF SHOWING ANTIMICROBIAL ACTIVITY AGAINST GRAM-NEGATIVE PATHOGENS

(71) Applicant: Rijksuniversiteit Groningen, CP Groningen (NL)

(72) Inventors: Qian LI, Wuhan (Hubei Province) (CN); Oscar Paul KUIPERS, Groningen (NL)

(21) Appl. No.: 16/622,533

(22) PCT Filed: Jun. 13, 2018
(86) PCT No.: PCT/NL2018/050385

§ 371 Date: Dec. 13, 2019

(30) Foreign Application Priority Data
Jun. 13, 2017 (EP) 17175773.5

(51) Int. Cl. A61K 38/16; A61K 38/08; A61K 38/10; A61P 31/04

(52) U.S. Cl. CPC A61K 38/164; A61P 31/04; A61K 38/10; A61K 38/08

(57) ABSTRACT

The invention relates to the field of medicine and microbiology, more specifically to means and methods for the treatment of infections caused by Gram-negative pathogens, in particular those showing or being prone to developing drug resistance. Provided is an admixture of (i) an inner membrane acting compound having membrane-permeating activity and/or lipid H binding activity; and (ii) one or more antimicrobial peptide(s) selected from the group consisting of RRLFRRIRWL-NH2 (GNP-6); GNNRPVYIPQPRPPHPRL (GNP-1); RIWVIWRR—NH2 (GNP-5); GIGKHVGKALKGLKGLLKGLGEC (GNP-7); and Xi X2IVQRIKKWLX3-NH2, wherein Xi is absent or K; X2 is R, K or A; and X3 is absent or R; wherein said one or more antimicrobial peptide(s) may comprise or consist of D- or L-amino acids.

The invention relates to the field of medicine and microbiology. More specifically, it relates to means and methods for the treatment of human or veterinary infections caused by Gram-negative pathogens, in particular those showing or being prone to developing drug resistance.

With the alarming resistance, which has now reached a critical point, there is a major and serious worldwide public health threat. Virtually no new broad- or small-spectrum antibiotics have been developed for the last half century, while the existing drugs are becoming ineffective rapidly[1]. Over the years, man became more and more reliant on antibiotics and even abused these drugs[1]. Unfortunately, the widespread use has no doubt increased the antibiotic resistance of bacterial[2, 3]. It has been pointed out in a review on 2016 that already 700,000 deaths per year because of spread of antimicrobial resistance (AMR), and this number is estimated to be 1,000,000 in future [4].

The World Health Organization (WHO) announced a report to give a global priority list of antibiotic-resistant bacteria to guide research, discovery and development of antibiotics[5]. 12 bacteria have been listed and the top 3 making up the category “critical” while 6 were classified as “high” and the other 3 were defined in “medium”. What is important, 9 of these 12 “superbugs” are Gram-negative pathogens while the 3 “critical” bacteria are all Gram-negative pathogens (Acinetobacter baumannii (carbapenem-resistant), Pseudomonas aeruginosa (carbapenem-resistant) and Enterobacteriaceae (carbapenem-resistant, 3rd generation cephalosporin-resistant).

Notably, the protective outer-membrane of Gram-negative bacteria functions as an efficient barrier to prevent several antimicrobials from reaching the cell membrane, which highly increases the level of difficulty of treatments towards multidrug-resistance (MDR) Gram-negative pathogens[6, 7]. To tackle this issue, there is an urgent need to search for new antibiotics or new therapeutic strategies against Gram-negative organisms.

Recognizing that the development of multidrug resistance pathogens has reached a crisis point, the present inventors sought to improve the management of existing drugs and to expand source for new drugs. More in particular, they aimed at providing novel compounds and therapeutic strategies that allow for adequately addressing the crisis of Gram-negative pathogenic bacteria.

It was surprisingly observed that specific peptides, herein further referred to as “Gram-negative outer membrane-perturbing peptides” (GNPs), can greatly enhance the anti-bacterial activity of known antimicrobials (e.g. nisin and vancomycin that commonly work best against Gram-positives)) against Gram-negative pathogens. As is shown herein below, selected GNPs were commercially synthesized and tested against Gram-negative pathogens either alone or combined with nisin or vancomycin. The results revealed that certain GNPs exerted a very effective synergistic effect towards 5 selected important Gram-negative pathogens when combined with nisin or vancomycin. Notably, the concentration of each compound that was needed to inhibit the growth of Gram-negative bacteria was dramatically reduced (up to 40 fold).

Overall, the approach disclosed in the present invention provides a highly promising way to expand the diversity of resources and strategies for the treatment of Gram-negative infections as well as increasing efficacy, decreasing the possible toxicity of antimicrobials and lower the rate of Gram-negative pathogens to become drug-resistant.

Accordingly, the invention provides an admixture of
(i) an inner membrane acting compound having membrane-permeating activity and/or lipid II binding activity; and
(ii) one or more antimicrobial peptide(s) selected from the group consisting of

(SEQ ID NO: 3)
RRLFRRILRWL-NH₂ (GNP-6)
(SEQ ID NO: 1)
GNNRPVYIPQPRPPHPRL (GNP-1)
(SEQ ID NO: 2)
RIWVIWRR-NH₂ (GNP-5)
(SEQ ID NO: 4)
GIGKHVGKALKGLKGLLKGLGEC (GNP-7);
and
(SEQ ID NO: 24)
X₁X₂IVQRIKKWLX₃R-NH₂ (GNP-8/-9 or GNP-8 mutant),

wherein X₁ is absent or K
X₂ is R, K or A
X₃ is absent or R;
   wherein said antimicrobial peptides may comprise or consist of D- or L-amino acids.

Among others, the invention provides an admixture of (i) an inner membrane or cytoplasmic acting compound; and (ii) one or more antimicrobial peptide(s) selected from the group consisting of GNNRPVYIPQPRPPHPRL (GNP-1) (SEQ ID NO:1); RIWVIWRR—NH₂ (GNP-5) (SEQ ID NO:2); RRLFRRILRWL-NH₂ (GNP-6) (SEQ ID NO:3); GIGKHVGKALKGLKGLLKGLGEC (GNP-7) (SEQ ID NO:4); RIVQRIKKWLR-NH₂ (GNP-8) (SEQ ID NO:15) and KRIVQRIKKWLR-NH₂ (GNP-9) (SEQ ID NO:16), wherein said antimicrobial peptides may comprise or consist of D- or L-amino acids.

An antimicrobial peptide of the invention shows a surprising synergy in the activity against Gram-negative pathogens in combination with an inner membrane acting compound having membrane-permeating activity and/or lipid II binding activity. In one embodiment, the inner membrane acting compound is an “inner membrane acting polypeptide”, which refers to a ribosomally or non-ribosomally synthesized peptide that commonly has membrane-permeating activity and/or lipid II binding activity, e.g. nisin or other lanthipeptides or derivatives thereof, or vancomycin or derivatives thereof, daptomycin, laspartomycin or macrolides. Examples include lantibiotics, like nisin, and lantibiotic derivatives thereof, and inner membrane acting antibiotic agents such as vancomycin. In another embodiment, the inner membrane acting compound belongs to the group of macrolides, which are a class of natural products that consist of a large macrocyclic lactone ring to which one or more deoxy sugars, usually cladinose and desosamine, may be attached. The lactone rings are usually 14-, 15-, or 16-membered. Macrolides belong to the polyketide class of natural products. Some macrolides have antibiotic or antifungal activity and are used as pharmaceutical drugs. Exemplary macrolides for use in the present invention include Azithromycin, Clarithromycin, Erythromycin, Fidaxomicin and Telithromycin.

In a further embodiment, the inner membrane acting compound is daptomycin or ciprofloxacin. Daptomycin is a lipopeptide antibiotic used in the treatment of systemic and life-threatening infections caused by Gram-positive organisms. It is a naturally occurring compound found in the soil saprotroph Streptomyces roseosporus. Its distinct mechanism of action makes it useful in treating infections caused by multiple drug-resistant bacteria. Other specific examples include daptomycin, laspartomycin, macrolides and ciprofloxacin.

It is known in the art that synergy can be observed between antimicrobial peptides. For example, Lüders et al. (Appl Environ Microbiol. 2003 March; 69(3): 1797-1799) investigated the antimicrobial effect obtained upon combining the prokaryotic antimicrobial peptides (AMPs; more commonly referred to as bacteriocins) pediocin PA-1, sakacin P, and curvacin A (all produced by lactic acid bacteria [LAB]) with the eukaryotic AMP pleurocidin from fish). It was found that the LAB AMPs and pleurocidin acted synergistically against a Gram-negative E. coli strain, albeit that the concentrations needed were still relatively high.

Van der Linden et al. (Biotechnology Letters 31(8):1265-7⋅May 2009) report a synergistic effect of an 51-residue ovine-derived cathelicidin and nisin against the Gram-positive S. aureus 1056 MRSA, but not against Gram-negatives.

Zhou et al. (2016 In: Frontiers in Cell and Developmental Biology. 4, p. 1-7 7 p., 7) investigated whether the activity of lantibiotics against Gram-negative bacteria could be improved by genetic fusion of several anti-Gram-negative peptides (e.g., apidaecin 1b, oncocin), or parts thereof, to the C-terminus of either full length or truncated nisin. It was found that when an eight amino acids (PRPPHPRL) tail from apidaecin 1b was attached to nisin, the activity of nisin against Escherichia coli CECT101 was increased more than two times.

In addition, each of the antimicrobial peptides, except for GNP-8 and mutants thereof, is known per se in the art. See Table 1. Importantly however, the synergistic effect of the defined GNPs when used in admixture (i.e. as physically distinct components) with an inner membrane acting polypeptide according to the present invention is not taught or suggested in the art.

TABLE 1
List of GNPs

| NName | Sequence | RReference | Source |
|---|---|---|---|
| GGN | GNNRPVYIPQPRPPHPRL | [[18] | Honey |
| P-1 | (SEQ ID NO: 1) | | bees |
| GGN | RIWVIWRR-NH₂ | [[22] | Bovine |
| P-5 | (SEQ ID NO: 2) | | |
| GGN | RRLFRRILRWL-NH₂ | [[23] | Synthetic |
| P-6 | (SEQ ID NO: 3) | | AMP.based |
| | | | on a cecropin |
| | | | A-melittin |
| | | | hybrid |
| GGN | GIGKHVGKALKGLKGLLKGL | [24] | Anuran |
| P-7 | GEC(SEQ ID NO: 4) | | |
| GGN | RIVQRIKKWLR-NH2 | TThis | Human |
| P-8 | (SEQ ID NO: 15) | work | (LL-37 |
| | | | derived |
| | | | fragment) |
| GGN | RIVQRIKKWL-NH₂ | This work | Human |
| P-8.1 | (SEQ ID NO: 17) | | (LL-37 |
| | | | derived |
| | | | fragment) |
| GGN | KIVQRIKKWLR-NH₂ | This work | Mutant human |
| P-8.2 | (SEQ ID NO: 18) | | |
| GGN | AIVQRIKKWLR-NH₂ | This work | Mutant human |
| P-8.3 | (SEQ ID NO: 19) | | |
| GGN | KRIVQRIKKWLR-NH2 | [25] | Human |
| P-9 | (SEQ ID NO: 16) | | |

In one embodiment of the invention, the inner membrane acting polypeptide is nisin or mutant or derivative thereof. Nisin has been used in food industry as natural-preservative for decades due to its high activity against bacteria and low toxicity for humans [8, 9]. In fact, after nisin reaches the bacterial plasma membrane, a pyrophosphate cage is formed via hydrogen bonds, which involves the first two rings of nisin and pyrophosphate of lipid II. The pyrophosphate cage is involved in the low resistance of bacteria to nisin because lipid II is an essential molecule which cannot change its nature (except for the pentapeptide moiety) and facilitates the transmembrane orientation of nisin [10, 11]. Nisin derivatives are known in the art. See for example engineered nisin derivatives nisin V and nisin I4V demonstrating enhanced functionalities (activity and/or stability) which make them more attractive from a clinical perspective (Cotter et al. (2013) Nat. Rev. Microbiol. 1195-105; Field et al., (2015) Bioengineered 6 187-192.) See also Field et al. (Front Microbiol. 2016 Apr. 18; 7:508) disclosing the potential of nisin and nisin derivatives to increase the efficacy of conventional antibiotics. In one embodiment, the nisin derivative is a fusion with either the full length or the truncated version of nisin containing the first three/five rings, e.g. as disclosed in Zhou et al. (2016 In: Frontiers in Cell and Developmental Biology. 4, p. 1-7 7 p., 7); Li et al. (Appl Environ Microbiol. 2018 May 31; 84(12) or MontalbAn-Lpez et al. FEMS Microbiol Rev. 2017 January; 41(1):5-18.

In another embodiment, the inner membrane acting polypeptide is vancomycin or a derivative thereof. Vancomycin is one of the most effective and safe medicines listed via WHO[12]. Vancomycin is a type of glycopeptide antibiotic, and the mechanism of it to kill Gram-positive bacteria is blocking the construction of cell wall[13]. Both nisin and vancomycin are very highly effective against Gram-positive bacteria with the minimal inhibitory concentrations (MICs) even at nanomolar levels[13-15]. However, their activity against Gram-negative bacteria is much lower. Exemplary vancomycin derivatives include dipicolyl-vancomycin conjugate (Dipi-van) and those disclosed in Yuki Nakama et al. (J. Med. Chem., 2010, 53 (6), pp 2528-2533) or WO2016/134622.

Exemplary nisin and vancomycin derivatives and mutants for use in the present invention are shown as highlighted in grey (similar activity as WT) or dark gray (increased activity) in Tables 2 and 3. In one embodiment, an admixture of the present invention comprises a nisin mutant selected from those shown in Table 2, preferably wherein the nisin mutant has an increased activity as compared to wildtype nisin. In another embodiment, an admixture of the present invention comprises a vancomycin derivative selected from those shown in Table 3, preferably wherein the vancomycin derivative has an increased activity as compared to unmodified vancomycin.

TABLE 2
Nisin A/Z mutants and their characteristics

| | | Biological | | |
|---|---|---|---|---|
| | | activity | | |
| Mutation | | (relative to the | | |
| name | Gene | wild type) | Characteristics | Ref |
| I1W | nisZ | Similar | Fluorescent label | [1] |
| | | activity | | |
| T2S | nisZ | Increased | Dha present in the final | [3] |
| | | activity | product instead of Dhb | |
| T2A | nisZ | Similar | Altering dehydrated residues | [3] |
| | | activity | | |
| T2V | nisZ | Similar | Altering dehydrated residues | [3] |
| | | activity | | |
| I4K/L6I | nisA | Similar | Altering residues in | [4] |
| | | activity | ring A of nisin A | |
| I4K/ | nisA | Increased | Altering residues in | [4] |
| S5F/L6I | | activity | ring A of nisin A | |
| K12T | nisA | Increased | Altering residue between | [8] |
| | | activity | ring A and ring B/C | |
| K12S | nisA | Increased | Altering residue between | [8] |
| | | activity | ring A and ring B/C | |
| K12N | nisA | Similar | Altering residue between | [8] |
| | | activity | ring A and ring B/C | |
| K12Q | nisA | Similar | Altering residue between | [8] |
| | | activity | ring A and ring B/C | |
| K12A | nisA | Increased | Altering residue between | [8] |
| | | activity | ring A and ring B/C | |
| K12P | nisA | Increased | Altering residue between | [8] |
| | | activity | ring A and ring B/C | |
| K12V | nisA | Increased | Altering residue between | [8] |
| | | activity | ring A and ring B/C | |
| K12M | nisA | Similar | Altering residue between | [8] |
| | | activity | ring A and ring B/C | |
| K12C | nisA | Similar | Altering residue between | [8] |
| | | activity | ring A and ring B/C | |
| K12L | nisA | Similar | Altering residue between | [8] |
| | | activity | ring A and ring B/C | |
| K12I | nisA | Similar | Altering residue between | [8] |
| | | activity | ring A and ring B/C | |
| K12P | nisZ | Similar | Positive charge reduction | [1] |
| | | activity | | |
| M17Q/ | nisZ | Similar | Altering residues in | [6] |
| G18T | | activity | ring C of nisin Z | |
| M17Q/ | nisZ | Similar | Altering residues in | [6] |
| G18Dh | | activity | ring C of nisin Z | |
| b | | | | |
| N20S | nisA | Similar | Altering residues | [10] |
| | | activity | in hinge region | |
| N20T | nisA | Similar | Altering residues | [10] |
| | | activity | in hinge region | |
| N20P | nisA | Increased | Altering residues | [10] |
| | | activity | in hinge region | |
| M21N | nisA | Similar | Altering residues | [10] |
| | | activity | in hinge region | |
| M21Q | nisA | Similar | Altering residues | [10] |
| | | activity | in hinge region | |
| M21G | nisA | Increased | Altering residues | [10] |
| | | activity | in hinge region | |
| M21A | nisA | Increased | Altering residues | [10] |
| | | activity | in hinge region | |
| M21S | nisA | Similar | Altering residues | [10] |
| | | activity | in hinge region | |
| M21T | nisA | Similar | Altering residues | [10] |
| | | activity | in hinge region | |
| M21V | nisA | Increased | Altering residues | [10] |
| | | activity | in hinge region | |
| M21I | nisA | Similar | Altering residues | [10] |
| | | activity | in hinge region | |
| M21K | nisA | Similar | Altering residues | [10] |
| | | activity | in hinge region | |
| K22Q | nisA | Similar | Altering residues | [10] |
| | | activity | in hinge region | |
| K22G | nisA | Increased | Altering residues | [10] |
| | | activity | in hinge region | |
| K22A | nisA | Increased | Altering residues | [10] |
| | | activity | in hinge region | |
| K22S | nisA | Increased | Altering residues | [10] |
| | | activity | in hinge region | |
| K22T | nisA | Increased | Altering residues | [10] |
| | | activity | in hinge region | |
| K22V | nisA | Similar | Altering residues | [10] |
| | | activity | in hinge region | |
| K22L | nisA | Similar | Altering residues | [10] |
| | | activity | in hinge region | |
| K22P | nisA | Similar | Altering residues | [10] |
| | | activity | in hinge region | |
| K22H | nisA | Similar | Altering residues | [10] |
| | | activity | in hinge region | |
| N20A/ | nisA | Similar | Altering residues | [11] |
| M21A/ | | activity | in hinge region | |
| K22A | | | | |
| M21A/K22I | nisA | Similar | Altering residues | [11] |
| | | activity | in hinge region | |
| N20F | nisZ | Similar | Altering residues | [12] |
| | | activity | in hinge region | |
| N20H | nisZ | Similar | Altering residues | [12] |
| | | activity | in hinge region | |
| N20K | nisZ | Similar | Altering residues in | [12] |
| | | activity | hinge region by | |
| | | | introducing positive charge | |
| N20Q | nisZ | Similar | Altering residues | [12] |
| | | activity | in hinge region | |
| N20V | nisZ | Increased | Altering residues | [12] |
| | | activity | in hinge region | |
| M21G | nisZ | Similar | Altering residues | [12] |
| | | activity | in hinge region | |
| M21H | nisZ | Similar | Altering residues | [12] |
| | | activity | in hinge region | |
| M21K | nisZ | Similar | Altering residues in | [12] |
| | | activity | hinge region by | |
| | | | introducing positive charge | |
| K22G | nisZ | Similar | Altering residues | [12] |
| | | activity | in hinge region | |
| K22H | nisZ | Similar | Altering residues | [12] |
| | | activity | in hinge region | |
| N20K/ | nisZ | Similar | Double mutation of | [12] |
| M21K | | activity | asparagine 20 and | |
| | | | methionine 21 to lysines | |
| N20F/ | nisZ | Similar | Hinge region of | [12] |
| M21L/ | | activity | nisinZ to hinge | |
| K22Q | | | region of subtilin | |
| N20A/ | nisZ | Increased | Hinge region of | [12] |
| M22K/ | | activity | nisinZ to hinge | |
| Dhb/ | | | region of epidermin | |
| K22G | | | | |
| N27K | nisZ | Similar | Charge alteration | [13] |
| | | activity | | |
| S29Q | nisA | Similar | Altering the residue | [14] |
| | | activity | at position 29 | |
| S29N | nisA | Similar | Altering the residue | [14] |
| | | activity | at position 29 | |
| S29D | nisA | Increased | Altering the residue | [14] |
| | | activity | at position 29 | |
| S29E | nisA | Increased | Altering the residue | [14] |
| | | activity | at position 29 | |
| S29A | nisA | Increased | Altering the residue | [14] |
| | | activity | at position 29 | |
| S29G | nisA | Similar | Altering the residue | [14] |
| | | activity | at position 29 | |
| S29L | nisA | Similar | Altering the residue | [14] |
| | | activity | at position 29 | |
| S29W | nisA | Similar | Altering the residue | [14] |
| | | activity | at position 29 | |
| S29M | nisA | Similar | Altering the residue | [14] |
| | | activity | at position 29 | |
| S29P | nisA | Similar | Altering the residue | [14] |
| | | activity | at position 29 | |
| I30W | nisA | Similar | Fluorescent label | [15] |
| | | activity | | |
| H31K | nisZ | Similar | Charge alteration | [13] |
| | | activity | | |
| NisA¹⁻³² | nisA | Similar | Proteolytically cleaved, | [17] |
| amide | | activity | all lanthionine ring present | |
| NisA1-34 | nisA | Increased | “PRPPHPRL” were | [18] |
| PRPPHPRL | | activity | added after nisin A | |
| NisA1-34 | nisA | Increased | “NGVQPKY” were | [19] |
| NGVQPKY | | activity | added after nisin A | |
| NisA1-28 | nisA | Increased | “SVNGVQPKYK” | [19] |
| SVNGVQPK | | activity | were added after ring | |
| YK | | | ABCDE of nisin A | |
| NisA1-28 | nisA | Increased | “SVKIAKVALKALK” | [19] |
| SVKIAKVA | | activity | were added after ring | |
| LKALK | | | ABCDE of nisin A | |

Note:
1) Increased activity, >120% compared to the activity of wild type nisin A/Z; Similar activity, 80%-100%
2) Mutants with Increased activity are labelled as bold; mutants with similar activity are labelled as normal font.
3) 5FW, 5-fluorotryptophan; 5HW, 5-hydroxytryptophan. Hinge region, amino acid residues between ring A/B/C and ring D/E; ΔN20/ΔM21, deletion of asparagine in position 20 and methionine in position21; Number in superscript, amino acid position.

TABLE 3
Vancomycin derivatives and their characteristics

| | Biological | | |
|---|---|---|---|
| | activity | | |
| | (relative | | |
| | to the | | |
| Derivative | wild type) | Characteristics | Ref |
| 1a | Increased | Bis (vancomycin) carboxamides, | [20] |
| | [symbol] | coupling of vancomycin | |
| | | with 1,6-diaminohexane | |
| 1b | Increased | Bis (vancomycin) carboxamides, | [20] |
| | [symbol] | coupling of vancomycin | |
| | | with cystamine | |
| 1c | Increased | Bis (vancomycin) carboxamides, | [20] |
| | [symbol] | coupling of vancomycin | |
| | | with homocystamine | |
| 2b | Increased | monomeric adducts of | [20] |
| | [symbol] | vancomycin with cystamine | |
| Siderophore- | Increased | Siderophore-vancomycin conjugates | [21] |
| vancomycin | [symbol] | | |
| A83850B | Similar | The amino sugar at amino acid 4 is | [22] |
| | activity | α -4-keto-L-epi-vancosamine. | |
| Compound | Increased | Chlorobiphenyl Vancomycin | [23] |
| 1 | activity | | |
| Compound | Increased | Chlorobiphenyl Des-methyl | [23] |
| 8 | activity | vancomycin | |
| 2a | Increased | Teicoplanin, contains | [24] |
| | activity | a hydrophobic substituent | |
| 3a | Increased | A hydrophobic substituent is | [24] |
| | activity | attached to the vancosamine | |
| | | nitrogen of vancomycin | |
| 4a | Increased | Derivatives containing | [24] |
| | activity | hydrophobic substituents | |
| | | on the glucose C6 position | |
| 5a | Increased | Derivatives containing | [24] |
| | activity | hydrophobic substituents | |
| | | on the glucose C6 position | |
| 3 | Increased | Covalent tail-to-tail dimers | [25] |
| | activity | of desleucyl vancomycin | |
| 4 | Increased | Covalent tail-to-tail dimers | [25] |
| | activity | of desleucyl vancomycin | |
| 5 | Increased | Corresponding intact dimers of 3 | [25] |
| | activity | | |
| 6 | Increased | Corresponding intact dimers of 4 | [25] |
| | activity | | |
| 4a | Similar | Vancomycin aglycon, | [26] |
| | activity | R = (Adam-1)CH₂NH | |
| 4c | Similar | Vancomycin aglycon, | [26] |
| | activity | R = H₂N(CH₂)₁₀NH | |
| 5 | Increased | Vancomycin-nisin(1-12) conjugate | [27] |
| | [symbol] | | |
| 6 | Similar | Vancomycin-nisin(1-12) conjugate | [27] |
| | activity ^{g} | | |
| 6 | Increased | Vancomycin-nisin(1-12) conjugate | [27] |
| | [symbol] | | |
| 7 | Increased | Vancomycin-nisin(1-12) conjugate | [27] |
| | [symbol] | | |
| Telavancin | Increased | A semi-synthetic derivative | [28] |
| | activity | of vancomycin that has a | |
| | | hydrophobic sidechain | |
| | | on the vancosamine sugar 2-4 | |
| 2 | Increased | Vancomycin aglycon, each | [30] |
| | [symbol] | of the four phenols were | |
| | | protected as methyl ethers | |
| 6 | Increased | Hydrophobic derivatives | [30] |
| | [symbol] | of vancomycin aglycon | |
| 7 | Increased | Hydrophobic derivatives | [30] |
| | [symbol] | of vancomycin aglycon | |
| 8 | Increased | Chlorobiphenyl vancomycin | [30] |
| | [symbol] | | |
| 2 | Similar | Phenyl group substituted | [31] |
| | activity ^{l,n,p} | derivative of vancomycin | |
| 3 | Similar | 4-methoxyphenyl-boronic acids | [31] |
| | activity ^{l,n,p} | substituted derivative of vancomycin | |
| 4 | Similar | 2-methoxyphenyl-boronic acids | [31] |
| | activity ^{l,n,p} | substituted derivative of vancomycin | |
| 5 | Similar | Styryl substituted derivative, | [31] |
| | activity ^{k,m,n,p} | 10-Dechloro-10-(trans- | |
| | | 2-phenylvinyl) vancomycin | |
| 5 | Increased | Styryl substituted derivative, | [31] |
| | [symbol] | 10-Dechloro-10-(trans- | |
| | | 2-phenylvinyl) vancomycin | |
| 6 | Similar | Styryl substituted derivative, | [31] |
| | activity ^{k,m,n,p} | 10-Dechloro-10-[trans-2-(4- | |
| | | methoxyphenyl)vinyl]vancomycin | |
| 6 | Increased | Styryl substituted derivative, | [31] |
| | [symbol] | 10-Dechloro-10-[trans-2-(4- | |
| | | methoxyphenyl)vinyl]vancomycin | |
| 7 | Similar | VanB-phe-notype derivative, | [31] |
| | activity ^{k,l,m,n} | 10-Dechloro-10-{trans- | |
| | | 2[4-(trifluoromethyl) | |
| | | phenyl]vinyl}lvancomycin | |
| 7 | Increased | VanB-phe-notype derivative, | [31] |
| | [symbol] | 10-Dechloro-10-{trans- | |
| | | 2-[4-(frifluoromethyl) | |
| | | phenyl]lvinyl}vancomycin | |
| 8 | Similar | Monooctenyl-substituted derivative, | [31] |
| | activity ^{k,l,n,o,p} | 10-Dechloro-10-(trans- | |
| | | oct-l-en-l-yl) vancomycin | |
| 9 | Similar | Monosubstituted derivative, | [31] |
| | activity ^{k,l,n,o,p} | 10-Dechloro-10-(trans-5-phenylpent- | |
| | | l-en-l-yl) vancomycin | |
| 10 | Increased | VanB-phe-notype derivative, | [31] |
| | [symbol] | 10-Dechloro-10-trans-[2-(biphenyl- | |
| | | 4-yl)vinyl]vancomycin | |
| 10 | Similar | VanB-phe-notype derivative, | [31] |
| | activity ^{l} | 10-Dechloro-10-trans-[2-(biphenyl- | |
| | | 4-yl)vinyl]vancomycin | |
| 11 | Similar | Dialkenyl-substituted derivatives, | [31] |
| | activity | 10,19-Didechloro-10,19-di- | |
| | | (trans-prop-l-en-l-yl) vancomycin | |
| 15 | Similar | G6-deoxy-vancomycin | [32] |
| | activity | | |
| 2 | Increased | Vancomycin aglycon | [33] |
| | [symbol] | | |
| 2 | Similar | Vancomycin aglycon | [33] |
| | activity ⁱ | | |
| 16 | Increased | Vancomycin aglycon (—Br) | [33] |
| | [symbol] | | |
| 29 | Increased | Vancomycin aglycon (—OH) | [33] |
| | [symbol] | | |
| 30 | Increased | Vancomycin aglycon (—H) | [33] |
| | [symbol] | | |
| 7 | Increased | Permethyl aglycon | [33] |
| | [symbol] | derivative (—CI) | |
| 7 | Similar | Permethyl aglycon derivative (—CI) | [33] |
| | activity ⁱ | | |
| 11b | Increased | Permethyl aglycon | [33] |
| | [symbol] | derivative (—B(OH)2) | |
| 14b | Increased | Permethyl aglycon | [33] |
| | [symbol] | derivative (—Br) | |
| 18b | Increased | Permethyl aglycon | [33] |
| | [symbol] | derivative (—NMe2) | |
| 19b | Increased | Permethyl aglycon | [33] |
| | [symbol] | derivative (—N3) | |
| 19b | Similar | Permethyl aglycon derivative (—N₃) | [33] |
| | activity ⁱ | | |
| 21b | Increased | Permethyl aglycon | [33] |
| | [symbol] | derivative (—CO2CH3) | |
| 22b | Increased | Permethyl aglycon derivative (—I) | [33] |
| | [symbol] | | |
| 23b | Increased | Permethyl aglycon | [33] |
| | [symbol] | derivative (—OMe) | |
| 24b | Increased | Permethyl aglycon | [33] |
| | [symbol] | derivative (—CH) | |
| 25b | Increased | Permethyl aglycon | [33] |
| | [symbol] | derivative (—H) | |
| 26b | Increased | Permethyl aglycon | [33] |
| | [symbol] | derivative (—OH) | |
| 27b | Increased | Permethyl aglycon | [33] |
| | [symbol] | derivative (—CH3) | |
| 28b | Increased | Permethyl aglycon | [33] |
| | [symbol] | derivative (—CF3) | |
| 9a | Increased | G6-Decanoyl- | [34] |
| | activity | vancomycin Derivative | |
| 9b | Increased | G4-Decanoyl- | [34] |
| | activity | vancomycin Derivative | |
| 9c | Increased | Z6-Decanoyl- | [34] |
| | activity | vancomycin Derivative | |
| 13 | Increased | G4-Octanoyl- | [34] |
| | activity | vancomycin Derivative | |

Note:
1) Increased activity, >120% compared with the activity of vancomycin; Similar activity, 80%-100.
2) Mutants with Increased activity was labelled as bold; mutants with similar activity was labelled as normal font.
3) Biological activity (relative to the wild type), letter in superscript for indicator strains:
a, 4 strains of E. faecium and E. faecalis exhibiting high-level resistance to vancomycin;
b, 10 vancomycin-susceptible strains of S.aureus;
c, Gram-positive organisms;
d, P.aerugmosa;
e, methicillin-resistant S.aureus;
f, vancomycin-susceptible strains of enterococci;
g, M.catarrhalis;
h, vancomycin-resistant strains of enterococci;
i, S.aureus;
j, E.faecali;
k, S. aureus (susceptible);
l, S. aureus (resistant);
m, E. faecium (susceptible);
n, E. faecium (resistant);
o, E. faecalis (susceptible);
p, E. faecalis (resistant).

In one embodiment of the invention, the admixture comprises an antimicrobial peptide selected from the group consisting of RRLFRRILRWL-NH₂ (GNP-6) (SEQ ID NO:3); GIGKHVGKALKGLKGLLKGLGEC (GNP-7) (SEQ ID NO:4); RIVQRIKKWLR-NH₂ (GNP-8) (SEQ ID NO:15); and KRIVQRIKKWLR-NH₂ (GNP-9) (SEQ ID NO:16).

The alpha amino acids are the most common form found in nature, but only when occurring in the L-isomer. The alpha carbon is a chiral carbon atom, with the exception of glycine which has two indistinguishable hydrogen atoms on the alpha carbon. Therefore, all alpha amino acids but glycine can exist in either of two enantiomers, called L or D amino acids, which are mirror images of each other. An antimicrobial peptide for use in the present invention may comprise or consist of L-amino acids or D-amino acids. For example, one or more “conventional” L-amino acids may be substituted by a D-amino acid. In one embodiment, the antimicrobial peptide consists of L-amino acids. In another embodiment, the antimicrobial peptide consists of D-amino acids. D-amino acids rarely occur naturally in organisms except for some bacteria. D-amino acids are highly resistant to protease-mediated degradation and have a low immunogenic response. This makes D-peptides especially interesting for use in an admixture of the invention.

Notably, as shown herein below, the D-GNPs exhibit an enhanced activity against Gram-negative pathogens when compared with the L-counterparts. In all cases the concentration is equal to the MIC of L-GNPs or reduced up to 4-fold. These data indicate that the selected GNPs do not specifically interact with a receptor.

In a specific aspect, the antimicrobial peptide is selected from the group consisting of RRLFRRILRWL-NH₂ (GNP-6) (SEQ ID NO:3), GIGKHVGKALKGLKGLLKGLGEC (GNP-7) (SEQ ID NO:4); RIVQRIKKWLR-NH₂ (GNP-8) (SEQ ID NO:15) and KRIVQRIKKWLR-NH₂ (GNP-9) (SEQ ID NO:16), wherein said antimicrobial peptide comprises or consists of D- or L-amino acids, preferably wherein said antimicrobial peptide consists of D-amino acids.

In a first aspect, the peptide is RRLFRRILRWL-NH₂ (SEQ ID NO:3) consisting of L-amino acids or consisting of D-amino acids. In a second aspect, the peptide is GIGKHVGKALKGLKGLLKGLGEC (SEQ ID NO:4) consisting of L-amino acids or consisting of D-amino acids. In a third aspect, the peptide is X₁X₂IVQRIKKWLX₃R—NH₂ (SEQ ID NO:24), wherein X₁ is absent or K; X₂ is R, K or A; and X₃ is absent or R, wherein said antimicrobial peptides may comprise or consist of D- or L-amino acids. In one embodiment, X₁ and/or X₃ is absent. In another embodiment, X₁ is K and/or X₃ is R. This can be combined with X₃ being either R, K or A, preferably wherein X₂ is R or K. For example, the peptide is RIVQRIKKWLR-NH₂ (GNP-8) (SEQ ID NO:15), KRIVQRIKKWLR-NH₂ (GNP-9) (SEQ ID NO:16), RIVQRIKKWL-NH₂ (GNP-8.1) (SEQ ID NO:17), KIVQRIKKWLR-NH₂ (GNP-8.2) (SEQ ID NO:18) or AIVQRIKKWLR-NH₂ (GNP-8.3) (SEQ ID NO:19) consisting of L-amino acids or consisting of D-amino acids.

Very good results are obtained wherein the antimicrobial peptide is RIVQRIKKWLR-NH₂ (GNP-8) (SEQ ID NO:15) comprising or consisting of D- or L-amino acids, preferably RIVQRIKKWLR-NH₂ (SEQ ID NO:15) consisting of D-amino acids, herein also referred to as “GNP-D8”. The FICI values resulting from the combinations of GNP-D8 and nisin/vancomycin ranged from 0.078 to 0.375. This suggested a very significant in vitro synergy. GNP-D8 was quite efficient to assist either nisin or vancomycin to reach the inner membrane.

As will be understood by a person skilled in the art, an admixture as provided herein may advantageously be supplemented with an additional antimicrobial agent. Also provided is a pharmaceutical composition comprising an admixture according to the invention, and a pharmaceutically acceptable vehicle, carrier or diluent.

A further aspect of the invention relates to an antimicrobial peptide of the amino acid sequence X₂IVQRIKKWLX-NH₂ (SEQ ID NO:15), wherein X₂ is R, K or A; and X₃ is absent or R, comprising or consisting of D- or L-amino acids, preferably consisting of D-amino acids. In one embodiment, the invention provides an antimicrobial peptide of the sequence RIVQRIKKWLR-NH₂ (GNP-8) (SEQ ID NO:15), RIVQRIKKWL-NH₂ (GNP-8.1) (SEQ ID NO:17), KIVQRIKKWLR-NH₂ (GNP-8.2) (SEQ ID NO:18) or AIVQRIKKWLR-NH₂ (GNP-8.3) (SEQ ID NO:19) comprising or consisting of D- or L-amino acids, preferably consisting of D-amino acids.

Such peptide is not disclosed or suggested in the art. US2015/0344527 teaches several antimicrobial peptides among which peptide 5 having the sequence RIVQRIKKWLLKWKKLGY (SEQ ID NO:9). Various short peptide analogs designed from human cathelicidin LL-37 are known in the art [25], including KR-12-a2 of the sequence KRIVQRIKKWLR-NH₂ (SEQ ID NO:16), thus having an additional N-terminal lysine and corresponding to microbial peptide GNP-9 as shown herein above. Surprisingly, it was observed that the absence of the lysine residue for specific pathogens increases the antimicrobial synergy when used in admixture with nisin. See Tables 4 and 5. Furthermore, it was found that replacing arginine at position 2 with lysine or alanine and/or deletion of the C-terminal arginine of peptides KR-12-a2/GMP-9 yields novel peptides showing a surprising antimicrobial activity (see Tables 10-12).

Hence, the invention also relates to a pharmaceutical composition comprising a peptide of the amino acid sequence X₂IVQRIKKWLX-NH₂ (SEQ ID NO:15), wherein X₂ is R, K or A; and X₃ is absent or R, comprising or consisting of D- or L-amino acids, preferably consisting of D-amino acids, and a pharmaceutically acceptable vehicle, carrier or diluent. In one embodiment, the invention provides a pharmaceutical composition comprising one or more of peptides RIVQRIKKWLR-NH₂ (GNP-8) (SEQ ID NO:15), RIVQRIKKWL-NH₂ (GNP-8.1) (SEQ ID NO:17), KIVQRIKKWLR-NH₂ (GNP-8.2) (SEQ ID NO:18) or AIVQRIKKWLR-NH₂ (GNP-8.3) (SEQ ID NO:19) comprising or consisting of D- or L-amino acids, preferably consisting of D-amino acids. RIVQRIKKWLR-NH₂ (SEQ ID NO:15) comprising or consisting of D- or L-amino acids, preferably consisting of D-amino acids, and a pharmaceutically acceptable vehicle, carrier or diluent.

Also provided is a bactericidal composition comprising a peptide of the amino acid sequence RIVQRIKKWLR-NH₂ (SEQ ID NO:15) comprising or consisting of D- or L-amino acids, preferably consisting of D-amino acids, optionally comprising one or more further antimicrobial agents; and excipients.

As will be appreciated by a person skilled in the art, a composition according to the invention is advantageously used in a method of preventing or treating a pathogenic infection in a subject, preferably a mammalian subject, more preferably a human subject. For example, the infection may be caused by a Gram-negative pathogen. In a specific embodiment, the infection is caused by a bacterium selected from the group consisting of E. coli, Klebsiella pneumoniae, Pseudomonas aeruginosa, Acinetobacter baumannii, Enterobacter cloaceae and Salmonella enterica. In one embodiment, the infection is caused by one or more pathogens known in the art as ESKAPE pathogens: Enterococcus faecium, Staphylococcus aureus, Klebsiella pneumoniae, Acinetobacter baumannii, Pseudomonas aeruginosa, and Enterobacter species.

A further advantageous aspect of the invention relates to the use of a peptide, admixture or composition in a method of preventing or treating a pathogenic infection in a subject, preferably a mammalian subject, more preferably a human subject, wherein the pathogenic infection is caused by a multi-drug resistant (MDR) bacterium, preferably an MDR bacterium of clinical relevance. For example, the infection is caused by one or more of the following bacteria:
Escherichia coli ATCC 25922
Escherichia coli ATCC BAA-2452
Escherichia coli B1927, clinical isolate
Klebsiella pneumoniae ATCC 700603
Klebsiella pneumoniae ATCC BAA-2524
Klebsiella pneumoniae B1945, clinical isolate
Pseudomonas aeruginosa ATCC 27853
Pseudomonas aeruginosa ATCC BAA-2108
Pseudomonas aeruginosa B1954, clinical isolate
Acinetobacter baumannii ATCC 17978
Acinetobacter baumannii ATCC BAA-1605
Acinetobacter baumannii B2026, clinical isolate

In a specific aspect, the invention provides the use of peptide GNP-D6 in a method of preventing or treating a pathogenic infection in a subject, preferably a mammalian subject, more preferably a human subject, wherein the pathogenic infection is caused by one or more of the following bacteria: E. coli ATCC BAA-2452, E. coli B1927, K. pneumoniae ATCC BAA-2524, K. pneumoniae B1945, P. aeruginosa ATCC BAA-2108, P. aeruginosa B1954 A. baumannii ATCC BAA-1605, A. baumannii B2026.

Also provided herein is a method to enhance the therapeutic potential and efficacy of an inner membrane acting compound against a Gram-negative pathogen, preferably wherein said inner membrane compound is selected from the group of nisin, vancomycin and functional derivatives thereof, comprising contacting said inner membrane acting compound with said Gram-negative pathogen in the presence of an antimicrobial peptide selected from the group consisting of RRLFRRILRWL-NH₂ (GNP-6) (SEQ ID NO:3); GNNRPVYIPQPRPPHPRL (GNP-1) (SEQ ID NO:1); RIWVIWRR—NH₂ (GNP-5) (SEQ ID NO:2); GIGKHVGKALKGLKGLLKGLGEC (GNP-7) (SEQ ID NO:4); RIVQRIKKWLR-NH₂ (GNP-8) (SEQ ID NO:15); RIVQRIKKWL-NH₂ (GNP-8.1) (SEQ ID NO:17); KIVQRIKKWLR-NH₂ (GNP-8.2) (SEQ ID NO:18); AIVQRIKKWLR-NH₂ (GNP-8.3) (SEQ ID NO:19); and KRIVQRIKKWLR-NH₂ (GNP-9) (SEQ ID NO:16), wherein said antimicrobial peptide(s) may comprise or consist of D- or L-amino acids.

A further aspect of the invention relates to the use of an antimicrobial peptides selected from the group consisting of GNNRPVYIPQPRPPHPRL (GNP-1) (SEQ ID NO:1); RIWVIWRR—NH₂ (GNP-5) (SEQ ID NO:2); RRLFRRILRWL-NH₂ (GNP-6) (SEQ ID NO:3); GIGKHVGKALKGLKGLLKGLGEC (GNP-7) (SEQ ID NO:4); RIVQRIKKWLR-NH₂ (GNP-8) (SEQ ID NO:15) and KRIVQRIKKWLR-NH₂ (GNP-9) (SEQ ID NO:16), wherein said antimicrobial peptide(s) may comprise or consist of D- or L-amino acids, to enhance the therapeutic potential and efficacy of an inner membrane acting polypeptide against a Gram-negative pathogen. The same preferences for the antimicrobial peptides and inner membrane acting polypeptide as disclosed herein above apply.

### LEGEND TO THE FIGURES

FIG. 1: Schematic picture of Synergy determination plate. A factorial dose matrix was used to trial all mixtures of the two serially diluted single compounds. No antibiotics were added to the wells of growth control while only medium was added in the wells of sterilization control.

FIG. 2: FICI of different combination against 5 different Gram-negative pathogens. Panels A-E: Nisin/Vancomycin+GNPs against different Gram-negative pathogens. (A)E. coli LMG15862; (B) K. pneumoniae; (C) P. aeruginosa LMG 6395; (D) A. baumannii LMG01041; (E). aerogenes LMG 02094. Panel F: Nisin+GNP-8/D8 and vancomycin+GNP-8/D8 against 5 Gram-negative pathogens. Different color correspond to different combination.

FIG. 3: Inhibition analysis of Nisin/vancomycin+GNP-8/GNP-D8 Panels A-E: Nisin/Vancomycin+GNPs against different Gram-negative pathogens. (A) E. coli LMG15862; (B) K. pneumoniae; (C) P. aeruginosa LMG 6395; (D) A. baumannii LMG01041; (E) E. aerogenes LMG 02094. Note: The actual start concentrations of nisin and peptide here are the MIC of nisin, vancomycin or GNPs when they are used alone against the Gram-negative pathogens inhibition effect of Nisin/vancomycin+GNP-8/GNP-D8 to the pathogens. We can see, there is very significant difference with control. In this combination which we used to calculate the FICI of antibiotics and GNPs, the growth of pathogens were completely inhibited. Note that although FICI of GNP-6/GNP-D6 are higher (slightly less synergistic effect) the actual absolute MIC values needed are even lower than those of GNP 8 or GNP-D8.

FIG. 4: Accurate concentration of nisin/vancomycin/GNPs when they were in combination tested against Gram-negative pathogens A-E: Nisin+GNPs against different Gram-negative pathogens A: indicator strain: E. coli LMG15862; B: indicator strain: K. pneumoniae; C: indicator strain: P. aeruginosa LMG 6395; D: indicator strain: A. baumannii LMG01041; E: indicator strain: E. aerogenes LMG 02094. F: Vancomycin+GNP8 against Gram-negative pathogens; G: Vancomycin+GNP-D8 against Gram-negative pathogens

### EXPERIMENTAL SECTION

### Materials and Methods for Examples 1-8

### 1.1. Material and Peptides

The purification and quantification of nisin was operated with HPLC as described previously [26]. Vancomycin was purchased from Sigma-Aldrich (Canada). Synthesized peptides were supplied by Proteogenix (France) and Pepscan (the Netherlands).

### 1.2. Bacterial Strains and Growth Conditions

The bacteria used in this study are given in Table 4. All the bacteria used were obtained from the Belgian Co-ordinated Collections of Micro-organisms (BCCM).

Escherichia coli, Klebsiella pneumoniae, Pseudomonas aeruginosa, Acinetobacter baumannii, Enterobacter aerogenes were grown in Luria-Bertani (LB) broth shaken (200 rpm) or on LB agar at 37° C. All of the strains were used to test the minimum inhibitory concentration (MICs) of nisin, vancomycin, synthesized peptides (GNPs) and combination test respectively.

TABLE 4
Strains and plasmids used

| | | | |
|---|---|---|---|
| Strains or Plasmids | Characteristics | Purpose | References |

Strains

| | | | |
|---|---|---|---|
| Escherichia coli | beta lactamase | Indicator strain | Lab |
| LMG15862 | | | collection |
| | | | BCCM |
| Klebsiella | beta lactamase | Indicator strain | Lab |
| pneumoniae | | | collection |
| LMG20218 | | | BCCM |
| Pseudomonas | | Indicator strain | Lab |
| aeruginosa LMG | | | collection |
| 6395 | | | BCCM |
| Acinetobacter | | Indicator strain | Lab |
| baumannii LMG | | | collection |
| 01041 | | | BCCM |
| Enterobacter | | Indicator strain | Lab |
| aerogenes LMG | | | collection |
| 02094 | | | BCCM |

### 1.3. Determination of Minimum Inhibitory Concentration (MICs)

MICs tests were performed in duplicate by a liquid growth inhibition microdilution assays in sterilized polypropylene microtiter plates according to Wiegand and Hilpert [27]. The indicator strains were first streaked on the appropriate agar plate and then incubated overnight. 3˜5 colonies were randomly picked and resuspended in saline (0.9% NaCl (w/v)) to make the OD₆₂₅ of the bacterial suspension≈0.08˜0.13 (12*10⁸ CFU/mL). The suspension was diluted in a ratio of 1:100 using Mueller Hinton Broth (MHB) and the final inoculum is 5*10⁵ CFU/mL. For each strain, row 11 with no peptide was included as growth control, while row 12 of medium-only wells was included as a sterility control. The antimicrobials were diluted serially by 1/2 one by one and 50 uL diluted bacterial suspension were added to each well to make the final volume to be 100 uL. Growth control was accomplished by remove 10 uL from well 11 and dilute 1000 times to plate 1/10 suspension on the solid medium. Microtiter plate and colonies count control were incubated at 37° C. for 16˜20 h without shaking, and growth inhibition was assessed measuring OD₆₀₀ using a microplate reader (Tecan infinite F200). The number of colonies on the control agar plate should be around 50 to ensure the concentration of the inoculum and the effectiveness of the experiment. The lowest concentration of the antimicrobials that inhibits visible growth of the indicator is identified as MIC.

### 1.4. Synergistic Effect Test of Nisin and GNPs

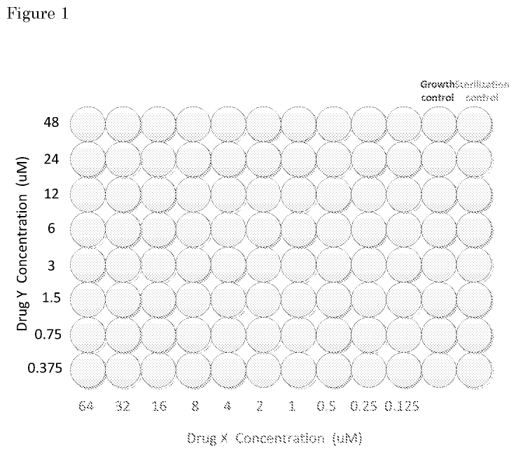

The test of synergistic effect was processed by conducting standard chequerboard broth microdilution assays [28, 29]. Nisin (Drug X) was loaded two fold serially diluted from row 1 to 10 in X-axis while GNPs (Drug Y) were loaded in eight two fold serially diluted concentrations from line A to H in Y-axis (FIG. 1). The original concentrations of peptides were MICs of each. Row 11 are used as growth control with no peptide was included, while row 12 with MHB medium-only wells was included as a sterility control as referred before. 50 uL fresh bacterial suspension was added to well 1-11 and final volume of each well on the plate is 100 uL.

### 1.5. Synergistic Effect Test of Vancomycin and GNPs

The test of synergistic effect was processed as method 1.4. Vancomycin and GNPs were two-fold serially diluted at X-axis and Y-axis separately.

### 1.6. Algorithm to Calculate Synergism

To determine whether the combination therapy is additive, synergistic or antagonistic, fractional inhibitory concentration (FIC) indices [30] were calculated. FICI=FICa+FICb=MICac/MICa+MICbc/MICb [28, 30]. The MICa/MICb is the MIC of compound A/B alone. MICac is the MIC of compound A in combination with compound B and MICbc is the MIC of compound B when it was combination with compound A. FIC is the MIC of compound alone divided by the MIC of compound in combination with the other compound. FICa is FIC of compound A while FICb is FIC of compound B. The FICI is interpreted as follows: synergistic, FICI≤0.5; additive, 0.5<FICI<1; indifferent, 1<FICI<2; antagonistic, FICI>2.

### Example 1: Activity Against Gram-Negative Pathogens of Nisin and L-Form GNPs Alone

Activity tests of nisin and GNPs based on L-amino acids were performed against five Gram-negative pathogens. The results of MICs are listed in Table 5. As it can be seen, MICs of GNPs were very different from each other among peptides and pathogens, and it varied from 2 μM to more than 256 μM. GNP-4 showed the lowest activity amongst all the GNPs while GNP5 and GNP6 are the most potent.

TABLE 5
MIC value of nisin and L-form GNP against Gram-negative pathogens

| Gram- | | | | | | | | | | |
|---|---|---|---|---|---|---|---|---|---|---|
| negative | Nisin | GNP-1 | GNP-2 | GNP-3 | GNP-4 | GNP-5 | GNP-6 | GNP-7 | GNP-8 | GNP-9 |
| pathogens | uM | uM | uM | uM | uM | uM | uM | uM | uM | uM |
| E. coli | 12 | 0.5 | 4 | 3 | 64 | 3 | 2 | 6 | 12 | 4 |
| LMG15862 | | | | | | | | | | |
| K. | 48 | 5 | 8 | 16 | >256 | 6 | 4 | 12 | 32 | 64 |
| pneumoniae | | | | | | | | | | |
| LMG20218 | | | | | | | | | | |
| P. | 36 | >64 | >64 | 32 | >128 | 3 | 3 | 13 | 16 | 16 |
| aeruginosa | | | | | | | | | | |
| LMG 6395 | | | | | | | | | | |
| A. | 6 | >64 | 16 | 8 | >128 | 2 | 2 | 2 | >64 | 16 |
| baumannii | | | | | | | | | | |
| LMG01041 | | | | | | | | | | |
| E. | 32 | 8 | >64 | >32 | >256 | 5 | 8 | 8 | 128 | 32 |
| aerogenes | | | | | | | | | | |
| LMG | | | | | | | | | | |
| 02094 | | | | | | | | | | |

### Example 2: Synergistic Effect of Nisin and L-Form GNPs in Admixture

The synergistic effect of nisin and GNPs were determined according to method 1.4. Although the MICs of the GNPs varied, all of them were combined with nisin and tested against E. coli. The results were listed in table 6. Two FICI of each combination are shown.

The FICI is interpreted as follows: synergistic, FICI≤0.5; additive, 0.5<FICI<1. Thus, we can conclude that nisin is additive with GNP-2 and GNP-3, while it is synergistic with all of the other peptides tested towards E. coli. The admixture of GNP-8+nisin appeared to be the best combination while the work concentration can be decreased to ¼ and 1/30 of the original concentration when used as separate antimicrobial agents.

The FICI of nisin+GNP4 was 0.375, which means that an admixture of 3 μM nisin and 8 μM GNP-4, or 1.5 μM nisin and 16 μM GNP-4 can completely inhibit the growth of E. coli. However, 8 μM or 16 μM is still a rather high concentration to be used in in vivo tests. Accordingly, in the following experiments to determine the synergistic effect, the focus is on peptides GNP-1, 5, 6, 7, and 8.

TABLE 6
Antimicrobial activity of admixtures of nisin
and L- form GNPs against E. coli LMG15862

| | Anti- | | MICa | MICb | MICac | MICbc | |
|---|---|---|---|---|---|---|---|
| Pathogen | biotic | GNP | (uM) | (uM) | (uM) | (uM) | FICI |
| E. coli | Nisin | GNP-1 | 12 | 0.5 | 1.5 | 0.13 | 0.375 |
| LMG15862 | | | | | 3 | 0.06 | 0.375 |
| | | GNP-2 | 12 | 4 | 1.5 | 2 | 0.625 |
| | | | | | 6 | 0.5 | 0.625 |
| | | GNP-3 | 12 | 3 | 3 | 1.5 | 0.75 |
| | | | | | 6 | 0.75 | 0.75 |
| | | GNP-4 | 12 | 64 | 3 | 8 | 0.375 |
| | | | | | 1.5 | 16 | 0.375 |
| | | GNP-5 | 12 | 3 | 0.75 | 0.75 | 0.313 |
| | | | | | 3 | 0.19 | 0.313 |
| | | GNP-6 | 12 | 2 | 3 | 0.25 | 0.375 |
| | | | | | 1.5 | 0.5 | 0.375 |
| | | GNP-7 | 12 | 6 | 3 | 0.75 | 0.375 |
| | | | | | 1.5 | 1.5 | 0.375 |
| | | GNP-8 | 12 | 12 | 3 | 0.38 | *0.281 |
| | | | | | 1.5 | 1.5 | *0.25 |
| | | GNP-9 | 12 | 4 | 3 | 0.13 | 0.281 |
| | | | | | 0.38 | 1 | 0.281 |

Italic font is used to highlight the MICac and MICbc which are both lower or close to 1 uM.
Bold: FICI > 0.5.
*the lowest FICI in this table.
Note:
MICa is MIC of nisin alone; MICb is GNP concentration when used alone; MICac is MIC of nisin in combination with the GNP at the MICbc concentration. MICbc is MIC of GNP when used with the MICac concentration of nisin.

The results of combination tests against K. pneumoniae, P. aeruginosa, A. baumannii and E. aerogenes are listed in table 7. Red was used to point out FICI which is lower than 0.1, and it illustrates that both concentration of nisin and GNPs are decreased at least 30 folds. The efficiency μM of admixtures was highest against K. pneumoniae and lowest against P. aeruginosa LMG 6395. The admixture of nisin+GNP-8 (1.5 μM nisin+0.5 μM GNP-8 or 0.75 μM nisin+1 μM GNP-8) and nisin+GNP-9 (3 μM nisin+1 μM GNP-9 or 0.75 μM nisin+4 μM GNP-9) are the combinations which indicate the best synergistic effect against K. pneumoniae. In these admixtures, the MICs of nisin and GNP-8 can be as low as 1/32 or 1/64 of the original's. Meanwhile, when P. aeruginosa and E. aerogenes were used as indicator strain, some admixtures were even shown to exert additive effect between two compounds. GNP-6 and GNP-7 appeared to act synergistically in admixture with nisin against all the 5 Gram-negative pathogens. In most cases, their MICs can be reduced to 4 to 8 times.

TABLE 7
Antimicrobial activity of nisin and L-form
GNPs against various Gram-negative bacteria

| | Anti- | | MICa | MICb | MICac | MICbc | |
|---|---|---|---|---|---|---|---|
| | biotic | GNP | (uM) | (uM) | (uM) | (uM) | FICI |
| K. | Nisin | GNP-1 | 48 | 5 | 12 | 0.63 | 0.375 |
| pneumoniae | | | | | 6 | 1.25 | 0.375 |
| LMG20218 | | GNP-5 | 48 | 6 | 12 | 0.75 | 0.375 |
| | | | | | 6 | 1.5 | 0.375 |
| | | GNP-6 | 48 | 4 | 3 | 0.5 | 0.188 |
| | | | | | 6 | 0.25 | 0.188 |
| | | GNP-7 | 48 | 12 | 3 | 0.38 | *0.094 |
| | | | | | 1.5 | 0.75 | *0.094 |
| | | GNP-8 | 48 | 32 | 1.5 | 0.5 | *0.047 |
| | | | | | 0.75 | 1 | *0.047 |
| | | GNP-9 | 48 | 64 | 3 | 1 | *0.078 |
| | | | | | 0.75 | 4 | *0.078 |
| P. | Nisin | GNP-1 | 36 | >64 | 4.5 | 64 | <1.13 |
| aeruginosa | | GNP-5 | 36 | 3 | 4.5 | 0.38 | 0.25 |
| LMG 6395 | | | | | 2.25 | 0.75 | 0.313 |
| | | GNP-6 | 36 | 3 | 4.5 | 0.75 | 0.375 |
| | | | | | 2.25 | 1.5 | [symbol] |
| | | GNP-7 | 36 | 13 | 2.25 | 6.5 | [symbol] |
| | | | | | 4.5 | 3.25 | 0.375 |
| | | GNP-8 | 36 | 16 | 2.25 | 8 | [symbol] |
| | | | | | 4.5 | 4 | 0.375 |
| | | GNP-9 | 36 | 16 | 4.5 | 0.5 | 0.156 |
| | | | | | 1.13 | 2 | 0.156 |
| A. | Nisin | GNP-1 | 6 | >64 | 1.5 | 64 | <1.25 |
| baumannii | | GNP-5 | 6 | 2 | 1.5 | 0.25 | 0.375 |
| LMG01041 | | | | | 0.75 | 0.5 | 0.375 |
| | | GNP-6 | 6 | 2 | 1.5 | 0.25 | 0.375 |
| | | | | | 0.75 | 0.5 | 0.375 |
| | | GNP-7 | 6 | 2 | 1.5 | 0.25 | 0.375 |
| | | | | | 0.75 | 0.5 | 0.375 |
| | | GNP-8 | 6 | >64 | 0.19 | 4 | *<0.094 |
| | | GNP-9 | 6 | 16 | 0.38 | 0.5 | *0.094 |
| | | | | | 0.19 | 1 | *0.094 |
| E. | Nisin | GNP-1 | 32 | 8 | 16 | 1 | [symbol] |
| aerogenes | | | | | 4 | 4 | [symbol] |
| LMG 02094 | | GNP-5 | 32 | 5 | 8 | 0.63 | 0.375 |
| | | | | | 4 | 1.25 | 0.375 |
| | | GNP-6 | 32 | 8 | 4 | 0.25 | 0.156 |
| | | | | | 1 | 1 | 0.156 |
| | | GNP-7 | 32 | 8 | 4 | 1 | 0.25 |
| | | | | | 8 | 0.5 | 0.313 |
| | | GNP-8 | 32 | 128 | 2 | 2 | 0.133 |
| | | | | | 4 | 1 | *0.078 |
| | | GNP-9 | 32 | >32 | 8 | 2 | <0.313 |
| | | | | | 2 | 8 | <0.313 |

Italics are used to highlight the MICac and MICbc which are both lower or close to 1 uM.
*FICI which is lower than 0.1, Bold and Italics: FICI which is higher than 0.5.

### Example 3: Activity of Admixtures of Vancomycin and D-Form GNPs Against Gram-Negative Pathogens

As demonstrated in the previous examples, peptides GNP6, GNP7, GNP8 and GNP9 exert a unique synergistic effect in admixture with nisin against Gram-negative pathogens. The sequences of peptides GNP8 and GNP9 are quite similar but GNP8 contains less net (positive) charge, which might be better absorbed by and utilized in the body. In this example, peptides GNP-6, GNP-7 and GNP-8 consisting solely of D-amino acids (referred to as GNP-D6, GNP-D7 and GNP-D8, respectively) were evaluated.

Vancomycin, an important and widely used inner membrane acting glycopeptide, was selected to test the synergistic effect with GNPs. Like nisin, vancomycin also contacts with cell membrane and inhibit the cell wall synthesis.

Table 8 shows the MICs of vancomycin and GNP-D6, GNP-D7 and GNP-D8 when used as individual agents against 5 Gram-negative pathogens. As predicted, vancomycin alone is not efficient against Gram-negative bacteria. The main reason could be the protective outer-membrane of Gram-negative pathogens which prevents vancomycin from reaching the inner-membrane. Surprisingly, the D-form GNPs were found to exhibit an enhanced activity against Gram-negative pathogens when compared with L-form GNPs.

TABLE 8
MIC value of vancomycin and D-form GNPs
against Gram-negative pathogens

| Gram-negative | Vancomycin | GNP-D6 | GNP-D7 | GNP-D8 |
|---|---|---|---|---|
| pathogens | μM | μM | μM | μM |
| E. coli LMG15862 | 64 | 2 | 2 | 4 |
| K. pneumoniae | 128 | 2 | 4 | 32 |
| LMG20218 | | | | |
| P. aeruginosa LMG | 128 | 2 | 4 | 16 |
| 6395 | | | | |
| A. baumannii | 32 | 2 | 2 | 8 |
| LMG01041 | | | | |
| E. aerogenes LMG | 192 | 2 | 4 | 32 |
| 02094 | | | | |

### Example 4: Synergistic Effect of Nisin in Admixture with D-Form GNPs

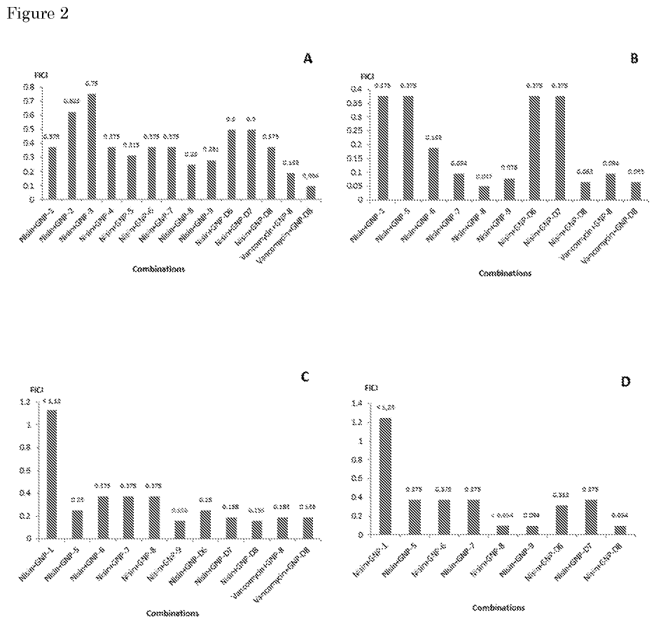

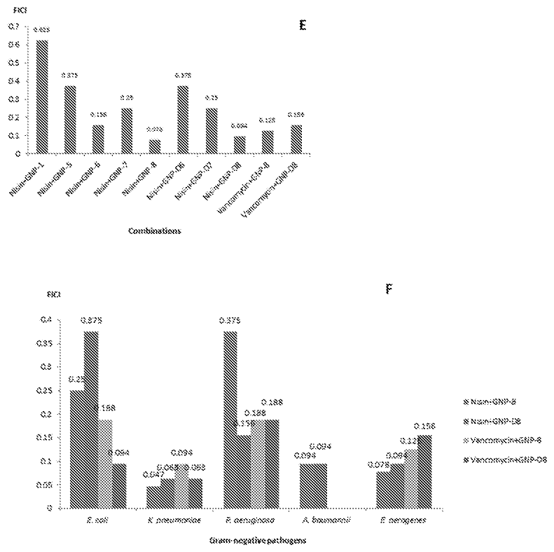

This Example demonstrates the antimicrobial activity of admixtures of nisin and D-form GNPs. As shown in Table 9, nisin+GNP-D8 shown a high efficiency against all 5 Gram-negative pathogens, with FICI values of K. pneumoniae, A. baumannii and E. aerogenes smaller than 0.1. When E. coli was used as indicator strain, the effect of GNP-6+nisin and GNP-7+nisin was shown to be additive. The FICI of GNP-D8 and nisin against K. pneumoniae is 0.063 or 0.073, while FICI of GNP-8+nisin is 0.047. These high levels of synergy are also observed when E. coli and E. aerogenes are used as indicator strains. Data analysis of the synergistic effect is shown in FIG. 2.

TABLE 9
Antimicrobial activity of admixtures of nisin and
D form GNPs against Gram-negative pathogens.

| | | | MICa | MICb | MICac | MICbc | |
|---|---|---|---|---|---|---|---|
| Antibiotic | Pathogen | GNP | (μM) | (μM) | (μM) | (μM) | FICI |
| Nisin | E. coli | GNP-D6 | 12 | 2 | 1.5 | 1 | [symbol] |
| | LMG15862 | | | | 3 | 0.5 | [symbol] |
| | | GNP-D7 | 12 | 2 | 1.5 | 1 | [symbol] |
| | | | | | 3 | 0.5 | [symbol] |
| | | GNP-D8 | 12 | 4 | 3 | 0.5 | 0.375 |
| | | | | | 1.5 | 1 | 0.375 |
| | K. | GNP-D6 | 48 | 2 | 12 | 0.25 | 0.375 |
| | pneumoniae | | | | 6 | 0.5 | 0.375 |
| | LMG20218 | GNP-D7 | 48 | 4 | 12 | 0.5 | 0.375 |
| | | | | | 6 | 1 | 0.375 |
| | | GNP-D8 | 48 | 32 | 0.75 | 2 | *0.078 |
| | | | | | 1.5 | 1 | *0.063 |
| | P. | GNP-D6 | 36 | 2 | 2.25 | 0.5 | 0.313 |
| | aeruginosa | | | | 4.5 | 0.25 | 0.25 |
| | LMG 6395 | GNP-D7 | 36 | 4 | 4.5 | 0.25 | 0.188 |
| | | | | | 2.25 | 0.5 | 0.188 |
| | | GNP-D8 | 36 | 16 | 4.5 | 0.5 | 0.156 |
| | | | | | 1.13 | 2 | 0.156 |
| | A. | GNP-D6 | 6 | 2 | 1.5 | 0.13 | 0.313 |
| | baumannii | | | | 0.38 | 0.5 | 0.313 |
| | LMG01041 | GNP-D7 | 6 | 2 | 0.75 | 0.5 | 0.375 |
| | | | | | 1.5 | 0.25 | 0.375 |
| | | GNP-D8 | 6 | 8 | 0.38 | 0.25 | *0.094 |
| | | | | | 0.19 | 0.5 | *0.094 |
| | E. | GNP-D6 | 32 | 2 | 8 | 0.25 | 0.375 |
| | aerogenes | | | | 4 | 0.5 | 0.375 |
| | LMG 02094 | GNP-D7 | 32 | 4 | 2 | 1 | 0.313 |
| | | | | | 4 | 0.5 | 0.25 |
| | | GNP-D8 | 32 | 32 | 2 | 1 | *0.094 |
| | | | | | 4 | 2 | 0.183 |

Italics are used to highlight the MICac and MICbc which are both lower or close to 1 uM.
*FICI which is lower than 0.1, Bold and Italics: FICI which is higher than 0.5.

### Example 5: Synergistic Effect of Vancomycin and GNP-8/GNP-D8 or GNP-6/GNP-D6

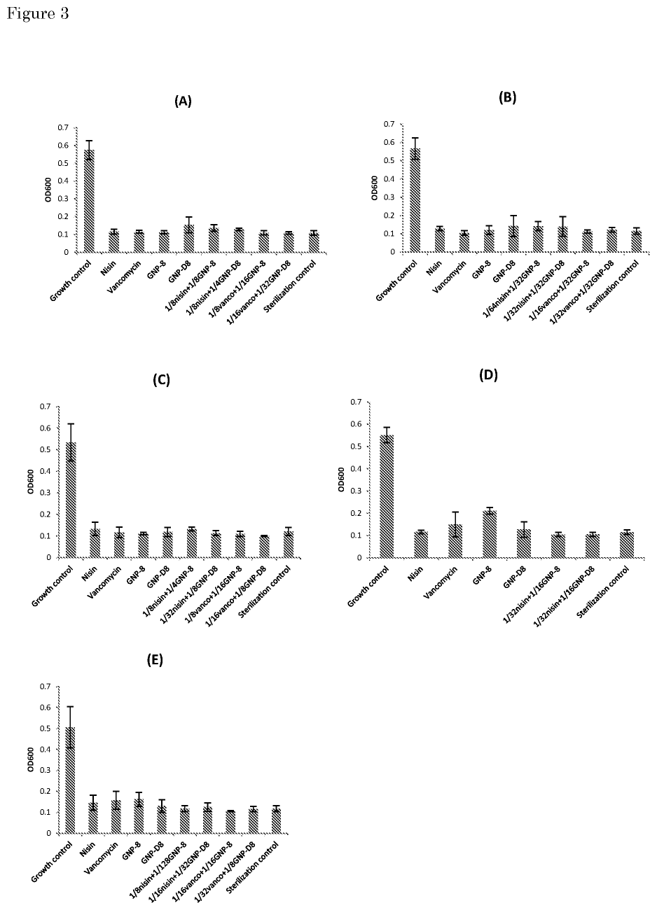

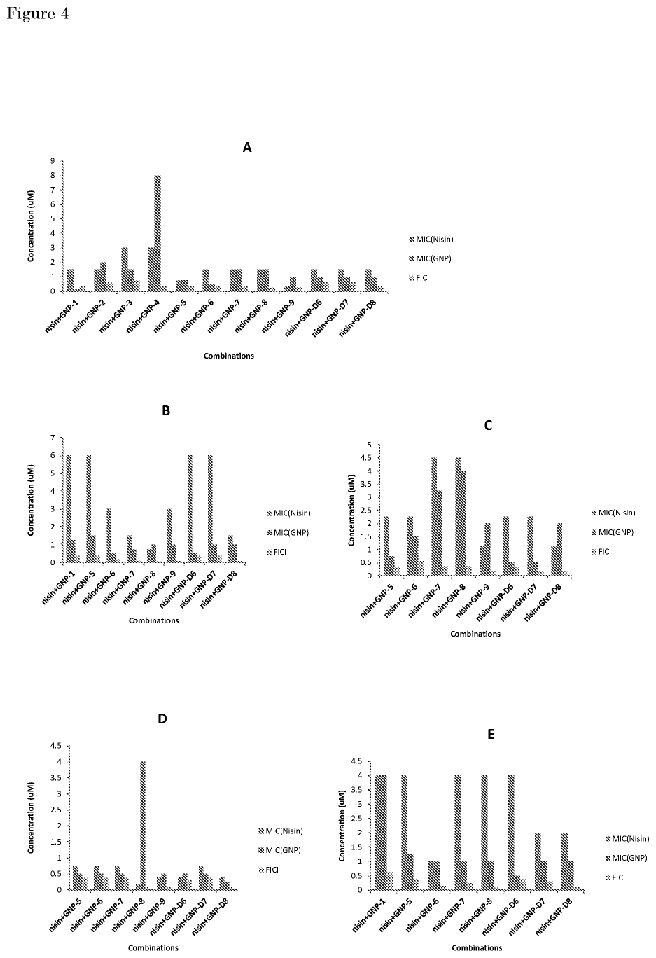

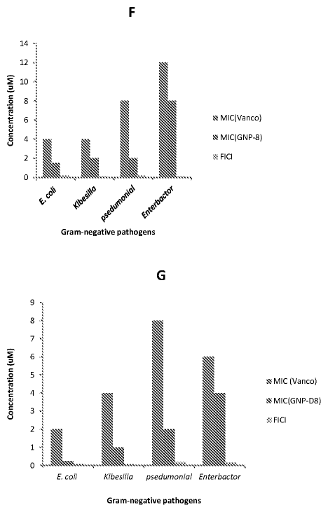

Antimicrobial tests were also performed with admixtures of vancomycin and GNPs. According to previous examples, GNP-8 and GNP-D8 performed very well in synergistic test. They were tested in admixture with vancomycin against 5 Gram-negative pathogens. As shown in table 10, both admixtures exhibited a highly synergistic effect against all the 5 Gram-negative pathogens. The concentration of both compounds declined to 1/32 to ⅛ of the MICs alone. The FICIs of K. pneumoniae are 0.094, which means MICs of both compounds are decreased 16 or 32 times at the same time. Data analysis of the synergistic effect is shown in FIG. 3. Table 11 shows the synergy observed for GNP-6/GNP-D6 and vancomycin.

TABLE 10
Combination activity of vancomycin and GNP-
8/GNP-D8 against Gram-negative pathogens

| | | | MICa | MICb | MICac | MICbc | |
|---|---|---|---|---|---|---|---|
| Antibiotic | Pathogen | GNP | (μM) | (μM) | (μM) | (μM) | FICI |
| Vanco- | E. coli | GNP-8 | 64 | 12 | 4 | 1.5 | 0.188 |
| mycin | LMG15862 | | | | 8 | 0.75 | 0.188 |
| | | GNP-D8 | 64 | 4 | 2 | 0.25 | *0.094 |
| | | | | | 4 | 0.125 | *0.094 |
| | K. | GNP-8 | 128 | 32 | 4 | 2 | *0.094 |
| | pneumoniae | | | | 8 | 1 | *0.094 |
| | LMG20218 | GNP-D8 | 128 | 32 | 8 | 1 | *0.094 |
| | | | | | 4 | 1 | *0.063 |
| | P. | GNP-8 | 128 | 16 | 8 | 2 | 0.188 |
| | aeruginosa | | | | 16 | 1 | 0.188 |
| | LMG 6395 | GNP-D8 | 128 | 16 | 8 | 2 | 0.188 |
| | | | | | 16 | 1 | 0.188 |
| | A. | GNP-8 | 32 | >64 | 0.5 | 4 | *<0.078 |
| | baumannii | | | | 2 | 1 | *<0.078 |
| | LMG01041 | GNP-D8 | 32 | 8 | 4 | 0.5 | 0.188 |
| | | | | | 2 | 1 | 0.188 |
| | E. | GNP-8 | 192 | 128 | 12 | 8 | 0.125 |
| | aerogenes | | | | 6 | 16 | 0.156 |
| | LMG 02094 | GNP-D8 | 192 | 32 | 12 | 4 | 0.188 |
| | | | | | 6 | 4 | 0.156 |

Italics are used to highlight the MICac decreases at least 16 folds when comparing to MICa and MICbc is lower or close to 1 uM.
*FICI which is lower than 0.1.
Note:
MICa: MIC of vancomycin alone; MICac: MIC of vancomycin in combination with GNP-8/GNP-D8.

TABLE 11
Combination activity of vancomycin and GNP-
6/GNP-D6 against Gram-negative pathogens

| | | | | | | | |
|---|---|---|---|---|---|---|---|
| Vanco- | | GNP-D6 | 64 | 2 | 16 | 1 | 0.75 |
| mycin | | | | | 16 | 0.5 | 0.5 |
| | K. pneumoniae | GNP-6 | 128 | 4 | 32 | 0.25 | 0.313 |
| | LMG20218 | | | | 16 | 0.25 | 0.25 |
| | | GNP-D6 | 128 | 2 | 32 | 0.5 | 0.5 |
| | | | | | 64 | 0.5 | 0.75 |
| | P. aeruginosa | GNP-6 | 128 | 3 | 32 | 1.5 | 0.75 |
| | LMG 6395 | GNP-D6 | 128 | 2 | 32 | 1 | 0.75 |
| | A. baumannii | GNP-6 | 32 | 2 | 8 | 0.5 | 0.5 |
| | LMG01041 | | | | 4 | 1 | 0.625 |
| | | GNP-D6 | 32 | 2 | 8 | 0.5 | 0.5 |
| | | | | | 4 | 1 | 0.625 |

Italics: FICI which is 0.5 or higher.

### Example 6: Synergistic Effect of GNP-8/GNP-9 Mutants and Nisin/Vancomycin

In this example, a set of six mutant peptides based on GNP-8/GNP-9 was designed, synthesized and tested. The sequences of mutants GNP-8.1, GNP8.2, GNP-8.3, GNP-8.4, GNP8.5 and GNP-8.6 are listed in table 12.

In mutant GNP8-1, the C-terminal arginine of GNP-8 was deleted to decrease the net charge of the peptide. The N-terminal arginine was replaced with lysine in mutant GNP8-2 and with alanine in mutant GNP8-3. Valine and glutamine were replaced by arginine and lysine to increase the hydrophilic and positive charges in GNP8-4 and GNP8-5. The order of arginine and lysine was reversed in GNP8-4 and GNP8-5. In mutant GNP8-6, the leucine residue in the C-terminus was deleted to yield a peptide with the same net charge but with a smaller spacing between the positively charged C-terminal amino acids.

TABLE 12
Amino acid sequence of GNP8/9 and mutants thereof

| | |
|---|---|
| GNP-8 | RIVQRIKKWLR-NH2 (SEQ ID NO: 15) |
| GNP-9 | KRIVQRIKKWLR-NH2 (SEQ ID NO: 16) |
| GNP8-1 | RIVQRIKKWL-NH2 (SEQ ID NO: 17) |
| GNP8-2 | KIVQRIKKWLR-NH2 (SEQ ID NO: 18) |
| GNP8-3 | AIVQRIKKWLR-NH2 (SEQ ID NO: 19) |
| GNP8-4 | RIRKRIKKWLR-NH2 (SEQ ID NO: 20) |
| GNP8-5 | RIKRRIKKWLR-NH2 (SEQ ID NO: 21) |
| GNP8-6 | RIVQRIKKWR-NH2 (SEQ ID NO: 22) |

The activity of each of nisin, vancomycin and de GNP-8 mutants alone against 5 Gram-negative pathogens is listed in Table 13, while the results of peptide admixtures with either nisin or vancomycin are shown in Tables 14 and 15. Vancomycin alone was quite modest against Gram-negative pathogens; at least 32 μM vancomycin was needed to inhibit the growth of the 5 selected Gram-negative pathogens. What is more, the MICs of vancomycin against K. pneumonia, P. aeruginosa and E. aerogenes were 128 μM, 128 μM and 192 μM, separately. Notably, the MICs of individual GNP-8 mutants were quite different from each other. Whereas GNP8-1, GNP8-2 and GNP8-3 showed a similar activity against certain bacteria when compared to GNP-8, the MICs of GNP8-4, GNP8-5 and GNP8-6 against K. pneumonia, A. baumannii and E. aerogenes were all above 256 μM. These results emphasize the importance of the core sequence IVQRIKKWL for the antimicrobial activity of the peptide.

TABLE 13
MIC value (μM) of antimicrobial compounds alone

| | | | | | E. | |
|---|---|---|---|---|---|---|
| | | K. | P. | A. | aerogenes | |
| | E. coli | pneumoniae | aeruginosa | baumannii | LMG | L. lactis |
| | LMG15862 | LMG20218 | LMG 6395 | LMG01041 | 02094 | MG1363 |
| Nisin | 12 | 48 | 36 | 6 | 32 | 0.006 |
| Vancomycin | 64 | 128 | 128 | 32 | 192 | 0.125 |
| GNP-6 | 2 | 4 | 3 | 2 | 8 | 1 |
| GNP-8 | 12 | 32 | 16 | >64 | 128 | 24 |
| GNP8-1 | 32 | 64 | 16 | 128 | >256 | ND |
| GNP8-2 | 16 | 32 | 16 | 128 | 256 | ND |
| GNP8-3 | 12 | 24 | 16 | 128 | 128 | ND |
| GNP8-4 | 16 | >256 | 16 | >256 | >256 | ND |
| GNP8-5 | 24 | >256 | 32 | >256 | >256 | ND |
| GNP8-6 | 256 | >256 | >256 | >256 | >256 | ND |
| GNP-D6 | 2 | 2 | 2 | 2 | 2 | 2 |
| GNP-D8 | 4 | 32 | 16 | 8 | 32 | 16 |

TABLE 14
Combined activity of nisin and GNPs
against 5 Gram-negative pathogens

| | | MICa | MICb | MICac | MICbc | |
|---|---|---|---|---|---|---|
| Pathogen | GNP | (μM) | (μM) | (μM) | (μM) | FICI |
| E. coli | GNP-6 | 12 | 2 | 1.5 | 0.5 | 0.375 |
| LMG15862 | GNP-8 | 12 | 12 | 1.5 | 1.5 | 0.25 |
| | GNP8-1 | 12 | 32 | 1.5 | 4 | 0.25 |
| | GNP8-2 | 12 | 16 | 1.5 | 2 | 0.25 |
| | GNP8-3 | 12 | 12 | 1.5 | 1.5 | 0.25 |
| | GNP8-4 | 12 | 16 | 3 | 2 | 0.375 |
| | GNP8-5 | 12 | 24 | 3 | 3 | 0.375 |
| | GNP8-6 | 12 | 256 | 6 | 64 | 0.75 |
| | GNP-D6 | 12 | 2 | 1.5 | 0.5 | 0.375 |
| | GNP-D8 | 12 | 4 | 1.5 | 1 | 0.375 |
| K. | GNP-6 | 48 | 4 | 3 | 0.5 | 0.188 |
| pneumoniae | GNP-8 | 48 | 32 | 0.75 | 1 | 0.047 |
| LMG20218 | GNP8-1 | 48 | 64 | 3 | 1 | 0.078 |
| | GNP8-2 | 48 | 32 | 0.75 | 2 | 0.078 |
| | GNP8-3 | 48 | 24 | 1.5 | 3 | 0.156 |
| | GNP8-4 | 48 | >256 | ND | ND | ND |
| | GNP8-5 | 48 | >256 | ND | ND | ND |
| | GNP8-6 | 48 | >256 | ND | ND | ND |
| | GNP-D6 | 48 | 2 | 6 | 0.5 | 0.375 |
| | GNP-D8 | 48 | 32 | 0.75 | 2 | 0.078 |
| P. | GNP-6 | 36 | 3 | 4.5 | 0.75 | 0.375 |
| aeruginosa | GNP-8 | 36 | 16 | 4.5 | 0.5 | 0.156 |
| LMG 6395 | GNP8-1 | 36 | 16 | 4.5 | 4 | 0.375 |
| | GNP8-2 | 36 | 16 | 4.5 | 4 | 0.375 |
| | GNP8-3 | 36 | 16 | 4.5 | 8 | 0.625 |
| | GNP8-4 | 36 | 16 | 2.25 | 4 | 0.313 |
| | GNP8-5 | 36 | 32 | 9 | 8 | 0.5 |
| | GNP8-6 | 36 | >256 | ND | ND | ND |
| | GNP-D6 | 36 | 2 | 4.5 | 0.25 | 0.25 |
| | GNP-D8 | 36 | 16 | 4.5 | 0.5 | 0.156 |
| A. | GNP-6 | 6 | 2 | 0.75 | 0.5 | 0.375 |
| baumannii | GNP-8 | 6 | >64 | 0.19 | 4 | <0.094 |
| LMG01041 | GNP8-1 | 6 | 128 | 0.375 | 16 | 0.188 |
| | GNP8-2 | 6 | 128 | 0.375 | 16 | 0.188 |
| | GNP8-3 | 6 | 128 | 0.375 | 32 | 0.313 |
| | GNP8-4 | 6 | >256 | ND | ND | ND |
| | GNP8-5 | 6 | >256 | ND | ND | ND |
| | GNP8-6 | 6 | >256 | ND | ND | ND |
| | GNP-D6 | 6 | 2 | 0.38 | 0.5 | 0.313 |
| | GNP-D8 | 6 | 8 | 0.19 | 0.5 | 0.094 |
| E. aerogenes | GNP-6 | 32 | 8 | 1 | 1 | 0.156 |
| LMG 02094 | GNP-8 | 32 | 128 | 4 | 1 | 0.078 |
| | GNP8-1 | 32 | >256 | ND | ND | ND |
| | GNP8-2 | 32 | 256 | 4 | 1 | 0.128 |
| | GNP8-3 | 32 | 128 | 4 | 8 | 0.188 |
| | GNP8-4 | 32 | >256 | ND | ND | ND |
| | GNP8-5 | 32 | >256 | ND | ND | ND |
| | GNP8-6 | 32 | >256 | ND | ND | ND |
| | GNP-D6 | 32 | 2 | 4 | 0.5 | 0.375 |
| | GNP-D8 | 32 | 32 | 2 | 2 | 0.125 |

Note:
Bold: the lowest FICI for the specific Gram-negative pathogen in this table; Italics: FICI >/= 0.5, ND: not determined.
Note:
MICa is the MIC of nisin alone; MICb corresponds to the MIC of GNPs when used alone; MICac is the MIC of nisin in combination with the GNP at the MICbc concentration. MICbc is the MIC of GNP when used with the MICac concentration of nisin.

TABLE 15
Combined activity of vancomycin and GNPs
against 5 Gram-negative pathogens

| | | MICa | MICb | MICac | MICbc | |
|---|---|---|---|---|---|---|
| Pathogen | GNP | (μM) | (μM) | (μM) | (μM) | FICI |
| E. coli | GNP-6 | 64 | 2 | 16 | 0.5 | 0.5 |
| LMG15862 | GNP-8 | 64 | 12 | 4 | 1.5 | 0.188 |
| | GNP8-1 | 64 | 32 | 8 | 4 | 0.25 |
| | GNP8-2 | 64 | 16 | 4 | 2 | 0.188 |
| | GNP8-3 | 64 | 12 | 4 | 3 | 0.313 |
| | GNP8-4 | 64 | 16 | 4 | 2 | 0.188 |
| | GNP8-5 | 64 | 24 | 4 | 3 | 0.188 |
| | GNP8-6 | 64 | 256 | 8 | 16 | 0.188 |
| | GNP-D6 | 64 | 2 | 16 | 0.5 | 0.5 |
| | GNP-D8 | 64 | 4 | 2 | 0.25 | 0.094 |
| K. | GNP-6 | 128 | 4 | 32 | 1 | 0.5 |
| pneumoniae | GNP-8 | 128 | 32 | 4 | 2 | 0.094 |
| LMG20218 | GNP8-1 | 128 | 64 | 16 | 8 | 0.25 |
| | GNP8-2 | 128 | 32 | 16 | 8 | 0.375 |
| | GNP8-3 | 128 | 24 | 16 | 3 | 0.25 |
| | GNP8-4 | 128 | >256 | ND | ND | ND |
| | GNP8-5 | 128 | >256 | ND | ND | ND |
| | GNP8-6 | 128 | >256 | ND | ND | ND |
| | GNP-D6 | 128 | 2 | 32 | 0.5 | 0.5 |
| | GNP-D8 | 128 | 32 | 8 | 1 | 0.094 |
| P. | GNP-6 | 128 | 3 | 32 | 1.5 | 0.75 |
| aeruginosa | GNP-8 | 128 | 16 | 8 | 2 | 0.188 |
| LMG 6395 | GNP8-1 | 128 | 16 | 16 | 2 | 0.25 |
| | GNP8-2 | 128 | 16 | 16 | 2 | 0.25 |
| | GNP8-3 | 128 | 16 | 4 | 8 | 0.531 |
| | GNP8-4 | 128 | 16 | 4 | 8 | 0.531 |
| | GNP8-5 | 128 | 32 | 16 | 8 | 0.5 |
| | GNP8-6 | 128 | >256 | ND | ND | ND |
| | GNP-D6 | 128 | 2 | 32 | 1 | 0.75 |
| | GNP-D8 | 128 | 16 | 8 | 2 | 0.188 |
| A. | GNP-6 | 32 | 2 | 8 | 0.5 | 0.5 |
| baumannii | GNP-8 | 32 | >64 | 2 | 1 | <0.078 |
| LMG01041 | GNP8-1 | 32 | 128 | 1 | 16 | 0.156 |
| | GNP8-2 | 32 | 128 | 4 | 4 | 0.156 |
| | GNP8-3 | 32 | 128 | 4 | 8 | 0.188 |
| | GNP8-4 | 32 | >256 | ND | ND | ND |
| | GNP8-5 | 32 | >256 | ND | ND | ND |
| | GNP8-6 | 32 | >256 | ND | ND | ND |
| | GNP-D6 | 32 | 2 | 8 | 0.5 | 0.5 |
| | GNP-D8 | 32 | 8 | 2 | 1 | 0.188 |
| E. aerogenes | GNP-6 | 192 | 8 | 8 | 2 | 0.291 |
| LMG 02094 | GNP-8 | 192 | 128 | 12 | 8 | 0.125 |
| | GNP8-1 | 192 | >256 | ND | ND | ND |
| | GNP8-2 | 192 | 256 | 12 | 16 | 0.125 |
| | GNP8-3 | 192 | 128 | 12 | 16 | 0.188 |
| | GNP8-4 | 192 | >256 | ND | ND | ND |
| | GNP8-5 | 192 | >256 | ND | ND | ND |
| | GNP8-6 | 192 | >256 | ND | ND | ND |
| | GNP-D6 | 192 | 2 | 32 | 1 | 0.667 |
| | GNP-D8 | 192 | 32 | 12 | 4 | 0.188 |

Bold: the lowest FICI for the specific Gram-negative pathogen in this table; Italics: FICI >/= 0.5; ND: not determined.
Note:
MICa is the MIC of vancomycin alone; MICb corresponds to the MIC of GNPs when used alone; MICac is the MIC of vancomycin in combination with the GNPs at the MICbc concentration. MICbc is the MIC of GNPs

### Example 8: Activity of Vancomycin, GNP-D6 and GNP-D8 Against PG Multidrug-Resistant Pathogens

Vancomycin, GNP-D6 and GNP-D8 were individually tested against several multi-drug resistant (MDR) Gram-negative pathogens (Table 16). The MIC values are listed in the Table 17.

TABLE 16
MDR Gram-negative pathogens used

| Strain | Characteristics |
|---|---|
| Escherichia coli ATCC | Clinical isolate, |
| BAA-2452 | MDR |
| Escherichia coli B1927 | Clinical isolate, |
| | MDR |
| Klebsiella pneumoniae | MDR |
| ATCC BAA-2524 | |
| Klebsiella pneumoniae | Clinical isolate, |
| B1945 | MDR |
| Pseudomonas aeruginosa | MDR |
| ATCC BAA-2108 | |
| Pseudomonas aeruginosa | MDR |
| B1954 | |
| Acinetobacter baumannii | MDR |
| ATCC BAA-1605 | |
| Acinetobacter | Clinical isolate, |
| baumannii | Colistin |
| B2026 | resistant |

TABLE 17
MIC value (μM) of MDR Gram-negative pathogens

| | Vancomycin | GNP-D6 | GNP-D8 |
|---|---|---|---|
| E.coli ATCC | >88.32 | 1.27 | 5.35 |
| BAA-2452 | | | |
| E. coli B1927 | 88.32 | 1.27 | 2.68 |
| K. pneumoniae | >88.32 | 2.53 | >85.62 |
| ATCC BAA-2524 | | | |
| K. pneumoniae B1945 | >88.32 | 5.05 | 21.41 |
| P. aeruginosa ATCC | >88.32 | 5.05 | >85.62 |
| BAA-2108 | | | |
| P. aeruginosa B1954 | 88.32 | 2.53 | 85.62 |
| A. baumannii ATCC | 44.16 | 5.05 | 10.7 |
| BAA-1605 | | | |
| A. baumannii B2026 | 22.08 | 2.53 | 10.7 |

The MIC values observed are consistent with those shown in Example 3.
GNP-D6 exerts a good activity when tested alone, while vancomycin and GNP-D8 show relatively high MIC value against the MDR Gram-negative pathogens.

### Example 8: Cell Toxicity and Hemolytic Activity of Vancomycin and GNP-D6/GNP-D8

Cellular toxicity of vancomycin, GNP-D6 and GNP-D8 were assessed using human HEK293 cells. ATP levels were measured by adding 50 μL of CellTiter-Glo reagent to each well and after 5 minutes of incubation luminescence was measured with SpectraMax i3. The effect on cell viability was determined by comparing the signal obtained in the presence of different concentrations of the compounds with those obtained in control wells without added compound. The effects were then calculated and presented as IC50 values and listed in Table 17.

It is shown that vancomycin and GNP-D8 do not affect the ATP levels at concentrations tested. The IC50 of GNP-D6 was 32.9 μM while the working concentration for inhibiting the growth of Gram-negative pathogens is always 2 μM (Table 10).

Fresh human red blood cells (HRBc) were used for the hemolytic tests. After incubation with different concentrations of test compounds at 37° C. for 1 hour, the absorbance of the supernatant were measured. The HC50 were listed in Table 18. Vancomycin and GNP-D8 did not induce hemolysis in the HRBc even at 500 μM and 300 μM, respectively. The HC50 of GNP-D6 was 168.9 μM, the latter being 80-fold higher than the working concentration of GNP-D6.

In conclusion, neither vancomycin and GNP-D8 cause lysis of human erythrocytes nor shows a significant toxicity against the human cell line HEK-239 at tested concentration. This indicates that vancomycin, GNP-D6 and GNP-D8 are safe to use and do not cause toxicity to human cells.

TABLE 18
Cell viability and hemolytic
activity of vancomycin,
GNP-D6 and GNP-D8.

| | IC₅₀ | HC₅₀ |
|---|---|---|
| | (uM) | (uM) |
| Vancomycin | >44.16 | >500 |
| GNP-D6 | 32.9 | 168.9 |
| GNP-D8 | 42.81 | >200 |

### Example 9: Antimicrobial Activity Against Clinically Relevant Gram-Negative Pathogens

In this example, the antibacterial activity of peptides GNP-D6 (“D6-peprtide”) and GNP-D8 (“D8-peptide”) is tested against 12 different bacterial strains alone and in combination with vancomycin using the established broth micro-dilution method. Testing was performed according to CLSI (Clinical Laboratory Standards Institute) guidelines. Read out of the study was determination of MIC—minimal inhibitory concentration expressed in μg/mL. In parallel, individual peptides, vancomycin and combinations thereof were tested in HEK293 cell line for their effect on cell viability.

Vancomycin and peptides alone were tested at concentrations starting from 128 μg/mL. Three different combinations of peptides with vancomycin were tested: vancomycin+D6 peptide in 1/0.3, 1/0.1 and 1/0.03 ratios; vancomycin and D8 peptide in 1/1, 1/0.3 and 1/0.1 ratios.

Bacterial strains tested were E. coli, K. pneumoniae, P. aeruginosa and A. baumannii strains—one ATCC quality control strain, one ATCC resistant strain and one clinical isolate for each type of bacteria.

### Materials and Methods

### 1.1. Materials

### 1.1.1. Test Compounds

Vancomycin hydrochloride, Sigma, V2002-250MG, Lot #037M4008V
D6 peptide
D8 (D-KR) peptide

### 1.1.2. Microbial Strains and Cells

Escherichia coli ATCC 25922
Escherichia coli ATCC BAA-2452
Escherichia coli B1927, clinical isolate
Klebsiella pneumoniae ATCC 700603
Klebsiella pneumoniae ATCC BAA-2524
Klebsiella pneumoniae B1945, clinical isolate
Pseudomonas aeruginosa ATCC 27853
Pseudomonas aeruginosa ATCC BAA-2108
Pseudomonas aeruginosa B1954, clinical isolate
Acinetobacter baumannii ATCC 17978
Acinetobacter baumannii ATCC BAA-1605
Acinetobacter baumannii B2026, clinical isolate
HEK293, ECACC, Cat. No. 85120602)

### 1.1.3. Culture Media and Equipment

BBL™ Mueller Hinton Broth, REF 275730, Lot. 7009699, Becton Dickinson
Mueller Hinton Agar 2, Ref. No. 97580-500G-F, Lot. No. BCBV4646, Sigma
Dulbecco's Modified Eagle's medium (DMEM), Cat. No. 41966-029, Gibco
Fetal bovine serum (FBS), Cat. No. F7524, Sigma
Non-essential amino acids (NEAA) 100x, Cat. No. 11140-035, Gibco
Phosphate-buffered saline (PBS) pH7.4 (10×), Gibco, Cat. No. 70011-036, Lot. No. 1972020
BioPhotometer, Eppendorf
SpectraMax i3 Microplate Reader, Molecular Devices

### 1.2. Methods

### 1.21. Peptides Preparation

For testing, 5 mg/mL solutions were prepared by dissolving peptide (solids) in PBS. Vancomycin was prepared by dissolving 5 mg in 1 mL of sterile water.

Out of these PBS solutions, 76.8 μL was transferred to 1423.2 μL of MH media in deep well for testing compound alone. For testing combinations, vancomycin and D6 peptide were mixed in 1:0.3, 1:0.1 and 1:0.03 ratios (76.8 μL of vancomycin+23.1 μL of D6+1400.1 μL of MH media; 76.8 μL of vancomycin+7.7 μL of D6+1415.5 μL of MH media; 76.8 μL of vancomycin+2.6 μL of D6+1420.6 μL of MH media) and vancomycin and D8 peptide in 1:1, 1:0.3 and 1:0.1 ratios (76.8 μL of vancomycin+76.8 μL of D8+1346.4 μL of MH media; 76.8 μL of vancomycin+23.1 μL of D8+1400.1 μL of MH media; 76.8 μL of vancomycin+7.7 μL of D8+1415.5 μL of MH media). Out of these working solutions 100 μL were transferred to wells in the third column of 96-well assay plates. Assay plates were previously filled with 50 μL of MH media in all wells except for the wells in the third column. Upon peptides and vancomycin addition, 50 μL was transferred from the third to the fourth column, then from the fourth to the fifth and so on. In this manner, the peptides and vancomycin were plated in 96-well assay plates in serial two-fold dilutions giving final concentrations range of 128-0.25 μg/mL.

### 1.2.2 Inoculum Preparation

Microorganisms used were all revived from skim milk storage at −70° C. by plating them on MH agar plates. The following day a single colony of each microorganism was again streaked on fresh agar plates. The next day, using direct colony suspension method, broth solutions that achieve turbidity equivalent to 0.5 McFarland standard for each microorganism were prepared. This has resulted in suspensions containing 1-2×10⁸ CFU/mL. Out of these suspensions, actual inoculums were prepared by diluting them 100× with MH media giving final microorganism count of 2-8×10⁵ CFU/mL. For each strain of microorganisms, 20 mL of these inoculum solutions were prepared. From the second to the twelfth column of 96-well plates, 50 μL of these solutions were transferred per well. To the first column, 50 μL per well of pure growth media was added. In this manner the first column was used as sterility control of media used, the second column was used as control of microorganism's growth and the rest of the plate was used for MIC determination. All plates were incubated for 16-24 h at 37° C.

### 1.2.3. MIC Determination

MIC value was determined by visual inspection of bacterial growth within 96-well plates. The first column in which there was no visible growth of bacteria was determined as MIC value for peptide or combination tested in that particular row.

### 1.2.4. Cell Viability Assessment

96-well plates were seeded with HEK293 cells at concentration of 30,000 cells per well in 100 μL of DMEM growth media supplemented with 1% NEAA and 10% FBS. Border wells were filled with 100 μL of sterile PBS. The next day compounds were added to cells. Compounds were first 2× diluted in 96-well V-bottom plate in PBS. After that compounds were mixed with media in 96-deep-well plate for final concentrations. Growth media from 3 plates was aspirated and replaced with 100 μL of prepared compounds. Compounds were tested in duplicate. ATP levels were measured by adding 50 μL of CellTiter-Glo reagent to each well and after 5 minutes of incubation luminescence was measured with SpectraMax i3. The potential effect of tested compounds on cell viability was determined by comparing the signal obtained in presence of different concentrations of the compounds with those obtained in control wells. The potential effects were then calculated and presented as IC₅₀ values (μg/mL).

### Results

The MIC values for tested peptides and combinations are given in Tables 19 and 20.

TABLE 19
MIC values for peptides and peptide combinations with vancomycin
(μg/mL) against E. coli and K. pneumoniae strains.

| | | E. coli | | K. | K. | |
|---|---|---|---|---|---|---|
| | E. coli | ATCC | | pneumoniae | pneumoniae | K. |
| | ATCC | BAA- | E. coli | ATCC | ATCC BAA- | pneumoniae |
| Compound(s) | 25922 | 2452 | B1927 | 700603 | 2524 | B1945 |
| Vancomycin | >128 | >128 | 128 | >128 | >128 | >128 |
| D6 | 2 | 2 | 2 | 4 | 4 | 8 |
| Van + D6 (1/0.3) | 8/2.4 | 8/2.4 | 8/2.4 | 8/2.4 | 8/2.4 | 16/4.8 |
| Van + D6 | 32/3.2 | 32/3.2 | 64/6.4 | 32/3.2 | 32/3.2 | 32/3.2 |
| (1/0.1) | | | | | | |
| Van + D6 | 64/1.9 | 64/1.9 | 64/1.9 | 128/3.8 | 128/3.8 | 128/3.8 |
| (1/0.03) | | | | | | |
| D8 | 8 | 8 | 4 | >128 | >128 | 32 |
| Van + D8 | 4/4 | 2/2 | 4/4 | 16/16 | 16/16 | 8/8 |
| (1/1) | | | | | | |
| Van + D8 | 8/2.4 | 4/1.2 | 8/2.4 | 32/9.7 | 16/4.8 | 16/4.8 |
| (1/0.3) | | | | | | |
| Van + D8 | 32/3.2 | 16/1.6 | 32/3.2 | 32/3.2 | 64/6.4 | 32/3.2 |
| (1/0.1) | | | | | | |

TABLE 20
MIC values for peptides and peptide combinations with vancomycin
(μg/mL) against P. aeruginosa and A. baumannii strains.

| | P. | P. | | A. | A. | |
|---|---|---|---|---|---|---|
| | aeruginosa | aeruginosa | P. | baumannii | baumannii | A. |
| | ATCC | ATCC BAA- | aeruginosa | ATCC | ATCC BAA- | baumannii |
| Compound(s) | 27853 | 2108 | B1954 | 17978 | 1605 | B2026 |
| Vancomycin | >128 | >128 | 128 | 128 | 64 | 32 |
| D6 | 4 | 8 | 4 | 2 | 8 | 4 |
| Van + D6 (1/0.3) | 16/4.8 | 32/9.7 | 16/4.8 | 8/2.4 | 8/2.4 | 4/1.2 |
| Van + D6 | 64/6.4 | 128/12.8 | 64/6.4 | 16/1.6 | 16/1.6 | 16/1.6 |
| (1/0.1) | | | | | | |
| Van + D6 | 128/3.8 | 128/3.8 | 128/3.8 | 64/1.9 | 32/1 | 32/1 |
| (1/0.03) | | | | | | |
| D8 | 64 | >128 | 128 | 16 | 16 | 16 |
| Van + D8 | 16/16 | 64/64 | 64/64 | 4/4 | 2/2 | 4/4 |
| (1/1) | | | | | | |
| Van + D8 | 64/19.4 | 128/38.8 | 128/38.8 | 8/2.4 | 4/1.2 | 8/2.4 |
| (1/0.3) | | | | | | |
| Van + D8 | 128/12.8 | >128/12.8 | 128/12.8 | 32/3.2 | 16/1.6 | 32/3.2 |
| (1/0.1) | | | | | | |

Table 21 shows the effects of the peptides and combinations of peptides with vancomycin on cell viability are given as ICbo values (μg/mL) for tested compounds in HEK293 cells.

TABLE 21
Effects of peptides and combination of peptides
with vancomycin on HEK293 viability

| Compound(s) | IC₅₀ (μg/mL) |
|---|---|
| Vancomycin | >64 |
| D6 | 52.1 |
| Van+D6 (1/0.3) | >64/19.4 |
| Van+D6 (1/0.1) | >64/6.4 |
| Van+D6 (1/0.03) | >64/1.9 |
| D8 | >64 |
| Van+D8 (1/1) | >64/64 |
| Van+D8 (1/0.3) | >64/19.4 |
| Van+D8 (1/0.1) | >64/6.4 |

These data show that cell toxicity is low for D6 and very low for D8.

### REFERENCES TO TABLE 2 AND 3

[1] Kuipers O P, Bierbaum G, Ottenwilder B, Dodd H M, Horn N, Metzger J, et al. Protein engineering of lantibiotics. Antonie Van Leeuwenhoek 1996; 69:161-70.
[3] Wiedemann I, Breukink E, van Kraaij C, Kuipers O P, Bierbaum G, de Kruijff B, et al. Specific binding of nisin to the peptidoglycan precursor lipid II combines pore formation and inhibition of cell wall biosynthesis for potent antibiotic activity. Journal of Biological Chemistry 2001; 276:1772-9.
[4] Rink R, Wierenga J, Kuipers A, Kluskens L D, Driessen A J, Kuipers O P, et al. Dissection and modulation of the four distinct activities of nisin by mutagenesis of rings A and B and by C-terminal truncation. Applied and environmental microbiology 2007; 73:5809-16.
[8] Molloy E M, Field D, Cotter P D, Hill C, Ross R P. Saturation mutagenesis of lysine 12 leads to the identification of derivatives of nisin A with enhanced antimicrobial activity. PloS one 2013; 8:e58530.
[10] Field D, Connor P M, Cotter P D, Hill C, Ross R P. The generation of nisin variants with enhanced activity against specific gram-positive pathogens. Mol Microbiol 2008; 69:218-30.
[11] Healy B, Field D, O'Connor P M, Hill C, Cotter P D, Ross R P. Intensive mutagenesis of the nisin hinge leads to the rational design of enhanced derivatives. PLoS One 2013; 8:e79563.
[12] Yuan J, Zhang Z-Z, Chen X-Z, Yang W, Huan L-D. Site-directed mutagenesis of the hinge region of nisinZ and properties of nisinZ mutants. Applied microbiology and biotechnology 2004; 64:806-15.
[13] Rollema H S, Kuipers O P, Both P, De Vos W M, Siezen R J. Improvement of solubility and stability of the antimicrobial peptide nisin by protein engineering. Applied and environmental microbiology 1995; 61:2873-8.
[14] Field D, Begley M, O'Connor P M, Daly K M, Hugenholtz F, Cotter P D, et al. Bioengineered nisin A derivatives with enhanced activity against both Gram positive and Gram negative pathogens. PLoS One 2012; 7:e46884.
[15] Martin I, Ruysschaerti J M, Sanders D, Giffard C J. Interaction of the lantibiotic nisin with membranes revealed by fluorescence quenching of an introduced tryptophan. The FEBS Journal 1996; 239:156-64.
[17] Chan W, Leyland M, Clark J, Dodd H, Lian L-Y, Gasson M, et al. Structure-activity relationships in the peptide antibiotic nisin: antibacterial activity of fragments of nisin. FEBS letters 1996; 390:129-32.
[18] Zhou L, van Heel A J, Montalban-Lopez M, Kuipers O P. Potentiating the Activity of Nisin against Escherichia coli. Frontiers in cell and developmental biology 2016; 4.
[19] Li Q, Montalban-Lopez M, Kuipers O P. Increasing Antimicrobial Activity of Nisin-based Lantibiotics Against Gram-negative Pathogens. Applied and environmental microbiology 2018:AEM. 00052-18.
[20] Sundram U N, Griffin J H, Nicas T I. Novel vancomycin dimers with activity against vancomycin-resistant enterococci. Journal of the American Chemical Society 1996; 118:13107-8.
[21] Malabarba A, Nicas T I, Thompson R C. Structural modifications of glycopeptide antibiotics. Medicinal research reviews 1997; 17:69-137.
[22] Nagarajan R Structure-activity relationships of vancomycin-type glycopeptide antibiotics. The Journal of antibiotics 1993; 46:1181-95.
[23] Ge M, Chen Z, Russell H, Kohler J, Silver L L, Kerns R, et al. Vancomycin derivatives that inhibit peptidoglycan biosynthesis without binding D-Ala-D-Ala. Science 1999; 284:507-11.
[24] Kerns R, Dong S D, Fukuzawa S, Carbeck J, Kohler J, Silver L, et al. The role of hydrophobic substituents in the biological activity of glycopeptide antibiotics. Journal of the American Chemical Society 2000; 122:12608-9.
[25] Jain R K, Trias J, Ellman J A. D-Ala-D-Lac binding is not required for the high activity of vancomycin dimers against vancomycin resistant enterococci. Journal of the American Chemical Society 2003; 125:8740-1.
[26] Printsevskaya S S, Solovieva S E, Olsufyeva E N, Mirchink E P, Isakova E B, De Clercq E, et al. Structure-activity relationship studies of a series of antiviral and antibacterial aglycon derivatives of the glycopeptide antibiotics vancomycin, eremomycin, and dechloroeremomycin. Journal of medicinal chemistry 2005; 48:3885-90.
[27] Arnusch C J, Bonvin A M, Verel A M, Jansen W T, Liskamp R M, de Kruijff B, et al. The vancomycin-nisin (1-12) hybrid restores activity against vancomycin resistant Enterococci. Biochemistry 2008; 47:12661-3.
[28] Saravolatz L D, Stein G E, Johnson L B. Telavancin: a novel lipoglycopeptide. Clinical infectious diseases 2009; 49:1908-14.
[30] Crane C M, Pierce J G, Leung S S, Tirado-Rives J, Jorgensen W L, Boger D L. Synthesis and evaluation of selected key methyl ether derivatives of vancomycin aglycon. Journal of medicinal chemistry 2010; 53:7229-35.
[31] Nakama Y, Yoshida O, Yoda M, Araki K, Sawada Y, Nakamura J, et al. Discovery of a novel series of semisynthetic vancomycin derivatives effective against vancomycin-resistant bacteria. J Med Chem 2010; 53:2528-33.
[32] Fowler B S, Laemmerhold K M, Miller S J. Catalytic site-selective thiocarbonylations and deoxygenations of vancomycin reveal hydroxyl-dependent conformational effects. Journal of the American Chemical Society 2012; 134:9755-61.
[33] Pinchman J R, Boger D L. Probing the role of the vancomycin E-ring aryl chloride: Selective divergent synthesis and evaluation of alternatively substituted E-ring analogues. Journal of medicinal chemistry 2013; 56:4116-24.
[34] Yoganathan S, Miller S J. Structure diversification of vancomycin through peptide-catalyzed, site-selective lipidation: a catalysis-based approach to combat glycopeptide-resistant pathogens. Journal of medicinal chemistry 2015; 58:2367-77.

### REFERENCES

[1] Gill E E, Franco O L, Hancock R. Chemical biology & drug design 2015; 85:56-78.
[2] Kristiansson E, Fick J, Janzon A, Grabic R, Rutgersson C, Weijdegard B, et al. PloS one 2011; 6:e17038.
[3] Piddock L J. The Lancet Infectious Diseases 2016.
[4] Tackling Drug-Resistant Infections Globally. 2016.
[5] Organization W H. Global Priority List of Antibiotic-Resistant Bacteria to Guide Research, Discovery, and Development of New Antibiotics. 2017.
[6] Santajit S, Indrawattana N. BioMed research international 2016; 2016.
[7] Helander I M, Von Wright A, Mattila-Sandholm T. Trends in Food Science & Technology 1997; 8:146-50.
[8] Delves-Broughton J, Blackburn P, Evans R, Hugenholtz J. Antonie Van Leeuwenhoek 1996; 69:193-202.
[9] Lubelski J, Rink R, Khusainov R, Moll G N, Kuipers O P. Cellular and molecular life sciences: CMLS 2008; 65:455-76.
[10] Breukink E, de Kruijff B. Nature reviews Drug discovery 2006; 5:321-3.
[11] Hsu S-T D, Breukink E, Tischenko E, Lutters M A, de Kruijff B, Kaptein R, et al. T. Nature structural & molecular biology 2004; 11:963-7.
[12] Organization W H. 19th WHO Model List of Essential Medicines. Cerca con Google 2015.
[13] Elyasi S, Khalili H, Dashti-Khavidaki S, Mohammadpour A. European journal of clinical pharmacology 2012; 68:1243-55.
[14] Li B, Yu J P, Brunzelle J S, Moll G N, van der Donk W A, Nair S K. Science 2006; 311:1464-7.
[15] AlKhatib Z, Lagedroste M, Zaschke J, Wagner M, Abts A, Fey I, et al. MicrobiologyOpen 2014; 3:752-63.
[16] Naghmouchi K, Baah J, Hober D, Jouy E, Rubrecht C, Sané F, et al. Antimicrobial agents and chemotherapy 2013; 57:2719-25.
[17] Des Field N S, Cotter P D, Ross R, Hill C. Frontiers in microbiology 2016; 7.
[18] Czihal P, Knappe D, Fritsche S, Zahn M, Berthold N, Piantavigna S, et al. ACS chemical biology 2012; 7:1281-91.
[19] Knappe D, Piantavigna S, Hansen A, Mechler A, Binas A, Nolte O, et al. Journal of medicinal chemistry 2010; 53:5240-7.
[20] Cudic M, Condie B A, Weiner D J, Lysenko E S, Xiang Z Q, Insug O, et al. Peptides 2002; 23:2071-83.
[21] Rao S S, Mohan K V, Atreya C D. PloS one 2013; 8:e56081.
[22] Spindler E, Hale J, Giddings T, Hancock R, Gill R. Antimicrobial agents and chemotherapy 2011; 55:1706-16.
[23] Torcato I M, Huang Y-H, Franquelim H G, Gaspar D, Craik D J, Castanho M A, et al. Biochimica et Biophysica Acta (BBA)-Biomembranes 2013; 1828:944-55.
[24] Ilid N, Novkovid M, Guida F, Xhindoli D, Benincasa M, Tossi A, et al. Biochimica et Biophysica Acta (BBA)-Biomembranes 2013; 1828:1004-12.
[25] Jacob B, Park I S, Bang J K, Shin S Y. Journal of Peptide Science 2013; 19:700-7.
[26] Zhou L, van Heel A J, Kuipers O P. Regulatory potential of post-translational modifications in bacteria 2015:100.
[27] Wiegand I, Hilpert K, Hancock R E. Nat Protoc 2008; 3:163-75.
[28] Stokes J M, MacNair C R, Ilyas B, French S, Côté J-P, Bouwman C, et al. Nature Microbiology 2017; 2:17028.
[29] Lehir J, Krueger A S, Avery W, Heilbut A M, Johansen L M, Price E R, et al. Nature biotechnology 2009; 27:659-66.
[30] Odds F C. Br Soc Antimicrob Chemo; 2003.

The content of the ASCII text file of the sequence listing named P115149PC00 Sequence Listing, having a size of 7.06 kb and a creation date of 21 Jul. 2020, and electronically submitted via EFS-Web on 22 Jul. 2020, is incorporated herein by reference in its entirety.

1. An admixture of
(i) an inner membrane acting compound having membrane-permeating activity and/or lipid II binding activity; and
(ii) one or more antimicrobial peptide(s) selected from the group consisting of
(SEQ ID NO: 3)
RRLFRRILRWL-NH₂ (GNP-6);
(SEQ ID NO: 1)
GNNRPVYIPQPRPPHPRL (GNP-1);
(SEQ ID NO: 2)
RIWVIWRR-NH₂ (GNP-5);
(SEQ ID NO: 4)
GIGKHVGKALKGLKGLLKGLGEC (GNP-7);
and
(SEQ ID NO: 24)
X₁X₂IVQRIKKWLX₃-NH₂,
wherein X₁ is absent or K
X₂ is R, K or A
X₃ is absent or R;
wherein said one or more antimicrobial peptide(s) may comprise or consist of D- or L-amino acids.

2. Admixture according to claim 1, wherein the antimicrobial peptide is selected from the group consisting of
(SEQ ID NO: 3)
RRLFRRILRWL-NH₂ (GNP-6)
(SEQ ID NO: 4)
GIGKHVGKALKGLKGLLKGLGEC (GNP-7)
(SEQ ID NO: 15)
RIVQRIKKWLR-NH₂ (GNP-8)
(SEQ ID NO: 17)
RIVQRIKKWL-NH₂ (GNP-8.1)
(SEQ ID NO: 18)
KIVQRIKKWLR-NH₂ (GNP-8.2)
(SEQ ID NO: 19)
AIVQRIKKWLR-NH₂ (GNP-8.3);
and
(SEQ ID NO: 16)
KRIVQRIKKWLR-NH₂ (GNP-9).

3. Admixture according to claim 1, wherein the one or more antimicrobial peptide(s) consists of L-amino acids.

4. Admixture according to claim 1, wherein the one or more antimicrobial peptide(s) consists of D-amino acids.

5. Admixture according to claim 1, wherein the antimicrobial peptide is selected from the group consisting of RRLFRRILRWL-NH₂ (GNP-6) (SEQ ID NO: 3), RIVQRIKKWLR-NH₂ (GNP-8) (SEQ ID NO: 15), RIVQRIKKWL-NH₂ (GNP-8.1) (SEQ ID NO: 17), KIVQRIKKWLR-NH₂ (GNP-8.2) (SEQ ID NO: 18), AIVQRIKKWLR-NH₂ (GNP-8.3) (SEQ ID NO:19) and KRIVQRIKKWLR-NH₂ (GNP-9) (SEQ ID NO:16).

6. Admixture according to claim 5, wherein the antimicrobial peptide is RRLFRRILRWL-NH₂ (GNP-6) (SEQ ID NO:3) or RIVQRIKKWLR-NH₂ (GNP-8) (SEQ ID NO:15) comprising or consisting of D- or L-amino acids.

7. Admixture according to claim 1, wherein the inner membrane acting compound is an inner membrane acting polypeptide.

8. Admixture according to claim 7, wherein the inner membrane acting polypeptide is nisin or vancomycin.

9. Admixture according to claim 1, wherein the inner membrane acting compound belongs to the group of macrolides.

10. Antimicrobial peptide of the sequence X₂IVQRIKKWLX₃—NH₂ (SEQ ID NO:15) wherein X₂ is R, K or A; and X₃ is absent or R, comprising or consisting of D- or L-amino acids.

11. Antimicrobial peptide according to claim 10 of the sequence RIVQRIKKWLR-NH₂ (GNP-8) (SEQ ID NO: 15), RIVQRIKKWL-NH₂ (GNP-8.1) (SEQ ID NO. 17), KIVQRIKKWLR-NH₂ (GNP-8.2) or AIVQRIKKWLR-NH₂ (GNP-8.3) (SEQ ID NO:19), comprising or consisting of D- or L-amino acids.

12. A bactericidal composition comprising a peptide according to claim 10, optionally comprising one or more further antimicrobial agent(s), and excipients.

13. A pharmaceutical composition comprising an admixture according to claim 1, and a pharmaceutically acceptable vehicle, carrier or diluent.

14. A composition according to claim 13, for use in a method of preventing or treating a pathogenic infection caused by a Gram-negative pathogen in a subject.

15. Composition for use according to claim 14, wherein said infection is caused by a bacterium selected from the group consisting of E. coli, Klebsiella pneumoniae, Pseudomonas aeruginosa, Acinetobacter baumannii, Enterobacter cloaceae and Salmonella enterica.

16. A method to enhance the therapeutic potential and efficacy of an inner membrane acting compound against a Gram-negative pathogen, comprising contacting said inner membrane acting compound with said Gram-negative pathogen in the presence of an antimicrobial peptide selected from the group consisting of
(SEQ ID NO: 3)
RRLFRRILRWL-NH₂ (GNP-6)
(SEQ ID NO: 1)
GNNRPVYIPQPRPPHPRL (GNP-1)
(SEQ ID NO: 2)
RIWVIWRR-NH₂ (GNP-5)
(SEQ ID NO: 4)
GIGKHVGKALKGLKGLLKGLGEC (GNP-7)
(SEQ ID NO: 15)
RIVQRIKKWLR-NH₂ (GNP-8)
(SEQ ID NO: 17)
RIVQRIKKWL-NH₂ (GNP-8.1)
(SEQ ID NO: 18)
KIVQRIKKWLR-NH₂ (GNP-8.2)
(SEQ ID NO: 19)
AIVQRIKKWLR-NH₂ (GNP-8.3);
and
(SEQ ID NO: 16)
KRIVQRIKKWLR-NH₂ (GNP-9),
wherein said antimicrobial peptide comprises or consist of D- or L-amino acids.

17. Method according to claim 16, wherein said inner membrane compound is nisin or vancomycin, or an active mutant or derivative thereof according to Table 2 or 3.
